(19) United States
(12) Patent Application Publication
(10) Pub. No.: US 2026/0345510 A1
(43) Pub. Date: Nov. 5, 2026

(54) MEMBRANE COATED NANOPARTICLES FOR DRUG DELIVERY

(71) Applicant: Arizona Board Regents on Behalf of Arizona State University, Tempe, AZ (US)

(72) Inventors: Kuei-Chun Wang, Gilbert, AZ (US); Hui-Chun Huang, Tempe, AZ (US); Ting-Yun Wang, Tempe, AZ (US); Joshua Rousseau, Fountain Hills, AZ (US)

(21) Appl. No.: 19/119,546

(22) PCT Filed: Oct. 13, 2023
(86) PCT No.: PCT/US2023/076904

§ 371 Date: Apr. 9, 2025

Related U.S. Application Data
(60) Provisional application No. 63/416,296, filed on Oct. 14, 2022.

(51) Int. Cl. A61K 9/51; A61K 31/405; A61K 31/409; A61K 31/506; A61K 31/575; A61P 9/10

(52) U.S. Cl. CPC A61K 9/5176; A61K 9/5192; A61K 31/405; A61K 31/409; A61K 31/506; A61K 31/575; A61P 9/10

(57) ABSTRACT

Provided herein are lipid nanoparticle complex with nanoparticles cloaked in monocyte plasma membrane for effective delivery of drugs. Also provided herein are drug loaded lipid nanoparticle complex for inhibition of YAP/TAZ pathway and methods of using the lipid nanoparticles for treatment of atherosclerosis in a subject in need thereof.

### CROSS-REFERENCE TO RELATED APPLICATIONS

The present application claims the benefit of U.S. Provisional Patent Application No. 63/416,296, entitled, “MEMBRANE COATED NANOPARTICLES FOR DRUG DELIVERY” filed Oct. 14, 2022, the content of which is hereby incorporated by reference in its entirety.

### GOVERNMENT SUPPORT

This invention was made with U.S. government support under R00 HL135416 awarded by the National Science Foundation. The U.S. government has certain rights in the invention.

### INCORPORATION OF SEQUENCE LISTING

The present application contains a Sequence Listing that which has been submitted in .XML format and is hereby incorporated by reference in its entirety. Said computer readable file was created on Oct. 13, 2023, is named 055743_770545_Sequence Listing.xml and is 8 kilobytes in size.

### FIELD

The present disclosure relates to targeted nanoparticle delivery using biomimetic nanocarriers and their use in site-specific inhibition of YAP/TAZ for atherosclerosis therapy.

### BACKGROUND

Atherosclerosis is a condition where plaque, which is a combination of fatty deposits, calcium, blood components, cells, and cholesterol, builds up on the inner walls of arteries throughout the body. As the plaque buildup increases, the affected artery or arteries narrows resulting in decreased blood flow through the affected area. Atherosclerosis is the primary cause of life-threatening cardiovascular events, and the effects of current therapies on lessening plaque burden remain far from optimal.

Although the causes of atherosclerosis are still under investigation, three different contributing factors have been identified in the buildup of plaque including arterial wall damage, inflammation, and high cholesterol levels. Symptoms of atherosclerosis typically present after one or more arteries are sufficiently blocked with plaque so that blood flow is significantly reduced possibly producing pain or discomfort. Unfortunately, in many persons, no symptoms present until an artery is completely blocked, often by a blood clot in a narrow artery, thereby causing a heart attack or stroke.

Additionally, delivery of drugs to blocked arteries can pose a problem. Maladaptive inflammation, a key feature in all stages of atherosclerosis, exacerbates plaque development and progression, prompting scientists to explore the potential of anti-inflammatory therapies for atherosclerosis. Recent clinical trials have demonstrated the feasibility of anti-inflammatory agents in reducing secondary cardiovascular events, positioning them as potential future therapeutic options for atherosclerosis, alongside statin treatments. Despite the success, the impact of systemic cytokine neutralization on plaque characteristics, as well as issues associated with impaired host defense and infections, remain to be addressed. In comparison to systemic treatments, local delivery of therapeutics that directly target specific inflammatory pathways in diseased blood vessels would be a more preferable approach to treat plaque progression, potentially improving effectiveness and safety. However, the complex nature of plaque makes the development of a lesion-targeted, pathway-specific strategy a highly challenging goal.

There is, therefore, a need for effective treatments and drug delivery methods to achieve site-specific inhibition of plaque and for treatment of atherosclerosis.

### SUMMARY

In some aspects the current disclosure encompasses a lipid nanoparticle complex comprising (a) a nanoparticle and (b) a lipid membrane encapsulating the nanoparticle. In some aspects, the lipid membrane comprises a monocyte plasma membrane. In some aspects, the lipid membrane does not comprise a cytosolic protein and comprises at least one membrane protein for example an adhesion protein, a glycoprotein or a type I transmembrane protein. In some aspects, the nanoparticles comprises at least one active pharmaceutical agent for example a YAP/TAZ inhibitor non-limiting examples of which include thiazovivin, cucurbitacin I, dasatinib, fluvastatin, pazopanib or verteporfin.

In some aspects, the lipid nanoparticle complex has an average hydrodynamic diameter of from about 200 to about 700 nm, about 300 to about 600 nm, or from about 200 to about 300 nm. In some exemplary aspects, the lipid nanoparticle complex has an average hydrodynamic diameter of about 280 nm. In some aspects, the lipid nanoparticle complex has a surface charge of about −15 to −20 mV, for example about −17.5 mV.

In some aspects, the current disclosure also encompasses pharmaceutical composition comprising a lipid nanoparticle as disclosed herein complex, and a carrier or excipient.

In some aspects, the current disclosure encompasses a method of producing a lipid nanoparticle complex, the method comprising (a) forming a nanoparticle comprising an active pharmaceutical agent, (b) isolating a plasma membrane from a monocyte, (c) contacting the plasma membrane of (b) with the nanoparticle of (a) to form the lipid nanoparticle complex.

In some aspects, the monocyte is isolated from a bone marrow of a subject. In some aspects, the nanoparticle comprises preparing a PLGA emulsion.

A method of treating atherosclerosis in a subject in need thereof, the method comprising administering to the subject an effective amount of a lipid nanoparticle of claim 1 or 2, or of the pharmaceutical composition of claim 13. In some aspects, the pharmaceutical composition is administered intravenously to atherosclerotic lesions. In some aspects the subject is a human.

In some aspects, the current disclosure also encompasses a lipid nanoparticle as disclosed herein, or a pharmaceutical composition as disclosed herein for use in the treatment of atherosclerosis.

### BRIEF DESCRIPTION OF THE FIGURES

FIG. 1A-1G show formulation and characterization of MoNP. FIG. 1A shows a schematic illustrating the preparation of MoNP loaded with a therapeutic payload. FIG. 1B shows flow cytometric analysis of Mo isolated from differentiated mouse BMC. FIG. 1C shows hydrodynamic size based on DLS results of MoNP, NP, and Mo vesicles. FIG. 1D shows PDI and surface charge based on DLS results of MoNP, NP, and Mo vesicles. Graphic data in FIG. 1D represent n=3, *p<0.05 vs. NP and #p<0.05 vs. Mo vesicles. FIG. 1E shows representative TEM images of MoNP, NP, and Mo vesicles. Scale bar=100 nm. FIG. 1F shows SDS-PAGE analysis of membrane protein profiles of MoNP and Mo vesicles. FIG. 1G shows western blot analysis of the membrane proteins of MoNP and Mo vesicles.

FIG. 2A-2B show in vitro stability of MoNP at different storage temperatures. Mo membrane-coated nanoparticles (MoNP) were resuspended in the storage buffer (10% sucrose in saline) and incubated at 25 °C, 4 °C, or −20 °C. FIG. 2A shows the change in hydrodynamic size of MoNP measured on day 1, 2, 3, and 4 after incubation using DLS analysis. FIG. 2B shows polydispersity index (PDI) of MoNP measured on day 1, 2, 3, and 4 after incubation using DLS analysis. n=3. *p<0.05 vs. 25 °C and #p<0.05 vs. 4 °C.

FIG. 3A-3C show physicochemical characterization of MoNP-DiD. FIG. 3A shows hydrodynamic size based on the DLS results of MoNP-DiD and NP-DiD. FIG. 3B shows surface charge based on the DLS results of MoNP-DiD and NP-DiD. FIG. 3C shows loading efficiency (LE) and encapsulation efficiency (EE) of MoNP-DiD. n=3-4, *p<0.05 vs. NP-DiD.

FIG. 4 shows in vitro payload release profiles of NP-DiD. The cumulative release profile of NP-DiD in saline pH 7, saline pH 7 containing 10% serum, or saline pH 6 for 96 hours was assessed. n=3.

FIG. 5A-5C show in vitro and in vivo biocompatibility assessment of MoNP. FIG. 5A shows numbers of live endothelial cells (EC) after incubating with MoNP or NP for 1, 2, and 3 days. n=3. ns indicates non-significance. FIG. 5B shows hemocompatibility assay of MoNP or NP resuspended in saline, were added to aliquots of mouse whole blood and incubated for 1 hour at 37 °C. After low-speed centrifugation, the tubes were imaged, and the absorbance at 540 nm was measured using a plate reader. Deionized water-added blood was used as a control group. *p<0.05 vs. water. FIG. 5C shows histological analysis of major organs isolated from the ApoE^{-/-} mice receiving 3 intravenous injections of MoNP or saline.

FIG. 6A-6F show MoNP enhanced endothelial uptake and lysosomal escape. FIG. 6A shows representative fluorescent images showing the cellular uptake of MoNP-DiD or NP-DiD by EC, TNFα-pretreated EC, TNFα-/anti-VCAM1-pretreated EC. FIG. 6B shows representative fluorescent images showing the cellular uptake of MoNP-DiD by EC under low or high SS. FIG. 6C shows representative fluorescent images showing the cellular uptake of MoNP-DiD or NP-DiD by Mo. FIG. 6D shows representative fluorescent images showing the cellular uptake of MoNP-DiD or NP-DiD by macrophages. Scale bar=100 μm. The intracellular DiD signal was quantified. For FIG. 6A, FIG. 6C, and FIG. 6D, n=3; *p<0.05 vs. NP-DiD, #p<0.05 vs. MoNP-DiD, $p<0.05 vs. MoNP-DiD/TNFα-stimulated EC. For FIG. 6B, n=3; *p<0.05 vs. low SS. FIG. 6E shows representative confocal images of EC incubated with MoNP-DiD or NP-DiD (red), followed by the staining of lysotracker (green) and nuclei (blue). Scale bar=25 μm. FIG. 6F shows correlation analysis of MoNP-DiD or NP-DiD with lysosomes. Thirteen cells were randomly selected from fluorescent images acquired in three biological repeats. *p<0.05 vs. NP-DiD.

FIG. 7A-7E show in vitro cellular uptake of MoNP by EC and phagocytes. FIG. 7A shows flow cytometry results showing the intracellular DiD signal in untreated EC, TNFα-pretreated EC, or TNFα-/anti-VCAM1-pretreated EC. FIG. 7B shows flow cytometry results showing the intracellular DiD signal in EC under high or low shear stress (SS). FIG. 7C shows flow cytometry results showing the intracellular DiD signal in Mo. FIG. 7D shows flow cytometry results showing the intracellular DiD signal in macrophages. FIG. 7E shows mouse whole blood was incubated with MoNP-DiD or NP-DiD at 37 °C with constant shaking at 120 rpm for 4 hours. After incubation, the samples were centrifuged to collect the plasma, and the fluorescent intensity was measured using a plate reader. n=3. *p<0.05 vs. NP-DiD.

FIG. 8A-8D show MoNP enabled active targeting of atheroprone arterial regions. FIG. 8A shows schematic showing the experimental design of MoNP-DiD or NP-DiD administration. FIG. 8B shows representative fluorescent images of the arterial tissues isolated from ApoE^{-/-} mice receiving MoNP-DiD or NP-DiD, with quantification of the fluorescent intensity measured in LCA, RCA, aortic arch (AA), and descending aorta (DA). Red: MoNP-DiD or NP-DiD. FIG. 8C shows representative fluorescent images of RCA and LCA cross-sections from the mouse receiving MoNP-DiD. Red: MoNP-DiD, green: CD31 and elastic layer autofluorescence, and blue: nuclei. The graph indicates the intensity of DiD signal of the crosslines (yellow). Scale bar=200 μm. FIG. 8D shows the fluorescent intensity of the major organs isolated from ApoE^{-/-} mice receiving MoNP-DiD or NP-DiD. n=6 for MoNP-DiD, and n=4 for NP-DiD. *p<0.05 vs. the NP-DiD group.

FIG. 9A-9G show MoNP-VP treatment alleviated the inflammatory response in EC. FIG. 9A is a schematic showing the experimental design of MoNP-VP treatment in EC. FIG. 9B shows physicochemical characterization of MoNP-VP. FIG. 9C shows western blot analysis of the TNFα-induced expression of VCAM1, ICAM1, YAP/TAZ, and CTGF in EC treated with MoNP-VP or MoNP. FIG. 9D shows qRT-PCR analysis of the TNFα-induced expression of VCAM1, ICAM1, and CTGF in EC treated with MoNP-VP or MoNP. For FIG. 9C and FIG. 9D, the data are normalized to its respective loading controls and the MoNP group. n=3; *p<0.05 vs. TNFα/MoNP. FIG. 9E shows representative images of fluorescently labeled Mo (green) attached to EC monolayers treated with MoNP-VP or MoNP. Scale bar=500 μm. n=3; *p<0.05 vs. TNFα/MoNP. FIG. 9F shows the RNA-Seq heatmap results displayed differences in the expression of atheroprone, atheroprotective, and YAP/TAZ-associated genes in TNFα-stimulated EC pretreated with MoNP-VP compared to those with MoNP. The genes are ranked based on their z-scores. FIG. 9G shows the KEGG pathway enrichment analysis of DEGs in response to MoNP-VP vs. MoNP in TNFα-stimulated EC. The inflammatory-related pathways and Hippo signaling pathway were highlighted in red. padj.<0.05.

FIG. 10A-10D show RNA-seq analysis of inflamed EC under MoNP-VP and MoNP treatments. FIG. 10A shows the volcano map showed 4,707 differentially expressed genes (DEGs) in TNFα/MoNP-verteporfin (VP)-treated EC vs. TNFα/MoNP-treated EC, including 2,333 upregulated genes and 2,374 downregulated genes. Magenta dots represent genes with |log₂FoldChange|>0.75 and p<0.05. The red nodes represent upregulated and downregulated DEGs. FIG. 10B depicts the heatmap of fold change for atheroprone, atheroprotective, and YAP/TAZ-associated genes in TNFα/MoNP-VP-treated EC vs. TNFα/MoNP-treated EC. FIG. 10C shows the top 30 enriched Gene Ontology (GO) terms of DEGs. FIG. 10D shows the Kyoto Encyclopedia of Genes and Genomes (KEGG) pathway enrichment cnetplot showing the enriched pathways of DEGs in response to MoNP-VP vs. MoNP in TNFα-treated EC.

FIG. 11A-11C show MoNP-VP treatment suppressed the PL-induced inflammatory response in vivo. FIG. 11A is a schematic showing the experimental design of MoNP-VP treatment (2 mg/kg) in ApoE^{-/-} mice. The arterial tissues were harvested 7 days after the PL procedure. FIG. 11B shows western blot analysis of the expressions of YAP/TAZ and CTGF in response to MoNP-VP or MoNP treatment. The band intensity is normalized to its respective loading controls. n=3; *p<0.05 vs. MoNP. FIG. 11C shows Immunofluorescence staining of YAP/TAZ, VCAM1, and CD68-positive cells in the arterial wall. Red: YAP/TAZ, VCAM1, or CD68, green: elastic layer autofluorescence, blue: nuclei. White asterisks indicate the lumen. The intensity of YAP/TAZ and VCAM1 in the intimal layer of LCA and the number of infiltrated CD68-positive cells were quantified. n=4, *p<0.05 vs. MoNP.

FIG. 12A-12H show MoNP-VP treatment attenuated plaque development in ApoE^{-/-} mice. FIG. 12A is a schematic showing the long-term treatment of MoNP-VP in mouse carotid atherosclerosis. ApoE^{-/-} mice were subjected to the PL procedure, followed by intravenous administration of MoNP-VP, free VP, MoNP, or saline after PL, and every 72 hours afterward, for a total of 6 injections. The lesion in the LCA was assessed on day 28 after PL. FIG. 12B shows representative images of the arterial tissues (from carotid bifurcation to DA) of various treatment groups. FIG. 12C shows representative images of the distal, middle, and proximal segments of the partially ligated LCA cross-sections from various treatment groups stained with Oil Red O and hematoxylin. FIG. 12D shows the Oil Red O-positive area. FIG. 12E shows the degree of luminal stenosis of the LCA. FIG. 12F shows the level of total cholesterol in mouse serum samples. FIG. 12G shows the changes in body weight of the mice throughout the experiment. FIG. 12H shows histological analysis of major organs isolated from ApoE^{-/-} mice subjected to various treatments. n=7 each for MoNP-VP, free VP, and MoNP, and n=5 for saline. *p<0.05 vs. saline, *p<0.05 vs. free VP, and $p<0.05 vs. MoNP.

FIG. 13A-13C show quantification of carotid atherosclerosis in ApoE^{-/-} mice under MoNP-VP treatment. FIG. 13A shows the partially ligated left carotid artery (LCA) was divided into three segments, the distal, middle, and proximal regions, starting from the carotid bifurcation. FIG. 13B shows quantitative analysis of Oil Red O-positive staining in the distal, middle, and proximal segments of LCA in ApoE^{-/-} mice injected with MoNP-VP, free VP, MoNP, or saline. FIG. 13C shows the degree of luminal stenosis of the LCA. n=7 each for MoNP-VP, free VP, and MoNP, and n=5. *p<0.05 vs. saline, #p<0.05 vs. free VP, and $p<0.05 vs. MoNP.

The drawing figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

The current disclosure is based in part, on the identification that Monocyte (Mo) membrane cloaking of nanoparticles by encapsulating nanoparticles with cell membranes from monocytes (MoNP) can enhance the ability for selective targeting and provide immune evasion of nanoparticles. Such MoNP can be effective biomimetic nanocarriers for targeted drug delivery and can increase the uptake of nanoparticles by inflamed EC, but not by phagocytic cells, and promote their accumulation in atheroprone vasculature. By utilizing MoNP to deliver VP, significant reductions in arterial YAP/TAZ expression, EC inflammation, macrophage infiltration, and plaque formation without causing significant organ toxicity can be undertaken. The disclosed MoNP are therefore a safe and effective strategy for treating diseases, for e.g., atherosclerosis.

In some aspects, the current disclosure encompasses compositions comprising these lipid nanoparticle complex comprising a nanoparticle and a lipid membrane and a pharmaceutical drug for therapeutic use. In some aspects, the current disclosure encompasses methods of making the membrane cloaked lipid nanoparticle complex disclosed herein. In some aspects, the current disclosure encompasses novel methods for targeted delivery of drugs using nanoparticles encapsulated with monocyte membrane and their use as biomimetic nanocarriers of drugs to achieve site-specific inhibition of YAP/TAZ for treatment of atherosclerosis.

### a. DEFINITIONS

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “atherosclerosis” refers to a condition where plaque, which is a combination of fatty deposits, calcium, blood components, cells, and cholesterol, builds up on the inner walls of arteries throughout the body. As the plaque buildup increases, the affected artery or arteries narrows resulting in decreased blood flow through the affected area. Atherosclerosis can lead to cardiovascular events. In some aspects, atherosclerosis disclosed herein can comprise atherosclerosis found in the cardiovascular and renal systems.

Atherosclerotic plaque generally grow slowly and over time may produce a severe stenosis (a narrowing of the diameter of the artery) or may progress to total arterial occlusion. With time, the plaque becomes calcified. Some plaques are stable, but others, especially those rich in lipids and inflammatory cells (e.g., macrophages) and covered by a thin fibrous cap, may undergo spontaneous fissure or rupture, exposing the plaque contents to flowing blood. These plaques are deemed to be unstable or vulnerable and are more closely associated to the onset of an acute ischemic event. The ruptured plaque stimulates thrombosis; the thrombi may embolize, rapidly occlude the lumen to precipitate a heart attack or an acute ischemic syndrome, or gradually become incorporated into the plaque, contributing to its stepwise growth.

Atherosclerosis is characteristically silent until critical stenosis, thrombosis, aneurysm, or embolus supervenes. Initially, symptoms and signs reflect an inability of blood flow to the affected tissue to increase with demand (e.g., angina or exertion, intermittent claudication). Symptoms and signs commonly develop gradually as the atheroma slowly encroaches on the vessel lumen.

“Pharmaceutical composition” means a mixture of substances suitable for administering to an individual that includes a pharmaceutical agent. As used herein a pharmaceutical composition comprises one or more of receptors, vectors, cells disclosed herein compounded with suitable pharmaceuticals carriers or excipients.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

As used herein, the term “patient”, “subject”, or “test subject” refers to any organism to which provided compound or compounds described herein are administered in accordance with the present disclosure, e.g., for experimental, diagnostic, prophylactic, and/or therapeutic purposes. Typical subjects include animals (e.g., mammals such as mice, rats, rabbits, non-human primates, humans). In an aspect, a subject is a human. In some aspects, a subject may be suffering from, and/or susceptible to a disease, disorder, and/or condition (e.g. atherosclerosis).

As used herein “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate (for e.g., a monkey and a chimpanzee).

The term “effective amount” as used herein is defined as the amount of the molecules of the present disclosure that are necessary to result in the desired physiological change in the cell or tissue to which it is administered. The term “therapeutically effective amount” as used herein is defined as the amount of the molecules of the present disclosure that achieves a desired effect with respect to atherosclerosis. A skilled artisan readily recognizes that in many cases the molecules may not provide a cure but may provide a partial benefit, such as alleviation or improvement of at least one symptom or parameter. In some aspects, a physiological change having some benefit is also considered therapeutically beneficial. Thus, in some aspects, an amount of molecules that provides a physiological change is considered an “effective amount” or a “therapeutically effective amount.”

As used herein, the disclosure of numerical ranges by numerical endpoints includes all numbers encompassed by that range (e.g., “1 to 5” includes but is not limited to 1, 1.25, 1.5, 1.75, 2, 2.3, 2.5, 2.8, 3, 3.1, 3.3, 3.8, 3.9, 4, 4.25, 4.5, 4.75 and 5). Unless otherwise indicated, all numbers used herein to express quantities, amounts, dimensions, measurements, and the like should be understood as encompassing the specific quantities, amounts, dimensions, measurements and so on, including those instances modified by the term “about.” For example, an amount disclosed herein as “about 1 mg/mL” would expressly include the amount of 1 mg/mL, and so on. Accordingly, unless indicated to the contrary, the numerical descriptions set forth herein may vary while remaining well within the teachings of the present disclosure. At the very least, each numerical value should be construed in view of the number of significant digits and by applying routine rounding techniques. As various changes could be made in the above-described cells and methods without departing from the scope of the disclosure, it is intended that all matter contained in the above description and in the examples given below, shall be interpreted as illustrative and not in a limiting sense.

As used herein, the term “nanoparticles” is defined as colloidal particles of sub-micron size of 10-1000 nanometers, such as 30-500 nanometers, or 50-350 nanometers, which may comprise a drug of interest entrapped into the matrix. Nanoparticles may be referred to as nanospheres, nanogels, nanocapsules, and micelles. The sub-micron size of nanoparticles has the advantages that they allow for cellular and tissue uptake, and they can pass through fine capillaries. Use of biodegradable materials in nanoparticle formulation may allow for sustained drug release at the target site over a period of weeks after injection. In some aspects, the nanoparticle disclosed herein can comprise an inner core which can be covered by an outer surface comprising the membrane as disclosed herein. The disclosure contemplates any nanoparticles now known and later developed that can be coated with the membrane described herein.

As used herein “plasma membrane” or “cellular membrane” refers to a biological membrane enclosing or separating structure acting as a selective barrier, within or around a cell, or a encloses a vacuole. The cellular membrane is selectively permeable to ions and organic molecules and controls the movement of substances in and out of cells. The cellular membrane comprises a phospholipid uni- or bilayer, and optionally associated proteins and carbohydrates. In some aspects, the plasma membrane refers to a membrane obtained from a naturally occurring biological membrane of a cell or cellular organelles, or one derived therefrom. As used herein, the term “naturally occurring” refers to one existing in nature. As used herein, the term “derived therefrom” refers to any subsequent modification of the natural membrane, such as isolating the cellular membrane, creating portions or fragments of the membrane, removing and/or adding certain components, such as lipid, protein or carbohydrates, from or into the membrane taken from a cell or a cellular organelle. A membrane can be derived from a naturally occurring membrane by any suitable methods. For example, a membrane can be prepared or isolated from a cell and the prepared or isolated membrane can be combined with other substances or materials to form a derived membrane. In another example, a cell or virus can be recombinantly engineered to produce “non-natural” substances that are incorporated into its membrane in vivo, and the cellular or viral membrane can be prepared or isolated from the cell or the virus to form a derived membrane.

As used herein “encapsulation” or “coating” or “cloaking” is interchangeably used, and refers to transferring biological membrane, for e.g., plasma membrane onto the surface of a nanoparticle.

As used herein “YAP/TAZ” refers to yes-associated protein (YAP) and transcriptional coactivator with PDZ-binding domain (TAZ) which are key regulators involved in the growth of whole organs, for amplification of tissue-specific progenitor cells during tissue renewal and regeneration, and for cell proliferation. YAP/TAZ can be used as therapeutic targets in cancer and regenerative medicine.

### b. MEMBRANE CLOAKED NANOPARTICLES

In some aspects, the current disclosure encompasses a lipid nanoparticle complex comprising a nanoparticle and a lipid membrane encapsulating the nanoparticle.

In some aspects, the nanoparticle disclosed herein is a non-metallic nanoparticle. In some aspects, the nanoparticles disclosed herein comprise a core-shell structure. In some aspects, the nanoparticles and/or the inner core of the nanoparticles comprises a polymer. In some aspects, the nanoparticles can be made from a wide range of materials. The nanoparticle is preferably composed of a material suitable for biological use. Examples of suitable nanoparticles include polystyrene nanoparticles, poly(lactic-co-glycolic) acid (PLGA) nanoparticles, PLURONICS stabilized polypropylene sulfide nanoparticles, and diamond particles. In other aspects, nanoparticles may be composed of glass, silica, polyesters of hydroxy carboxylic acids, polyanhydrides of dicarboxylic acids, or copolymers of hydroxy carboxylic acids and dicarboxylic acids. In some aspects, the nanoparticles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, the nanoparticles can be quantum dots, or composed of quantum dots, such as quantum dot polystyrene nanoparticles. Nanoparticles including mixtures of ester and anhydride bonds (e.g., copolymers of glycolic and sebacic acid) may also be employed. For example, nanoparticles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), [rho]oly(lactic-co-sebacic) acid copolymers (PLSA), poly(glycolic-co-sebacic) acid copolymers (PGSA), polypropylene sulfide polymers, poly(caprolactone), collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polypeptides, proteins, polysaccharides, hyaluronic acid and alginate, acyl-substituted cellulose acetates, polyethylene oxide, glycerin, sorbitol, mannitol, sucrose, sorbitan, glycerol, xylitol, isomalt, polypropylene glycol, and poly(tetramethylene ether)glycol, polycaprolactones or polyester adipate polyols, polyether polyols, trehalose, lactose, glucose, or dextran, as well as biopolymers known in the art. Other biocompatible, biodegradable polymers useful in the present disclosure include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. In some aspects, the particle is biodegradable in an individual. In such aspects, the nanoparticles can be provided in an individual across multiple doses without there being an accumulation of nanoparticles in the individual. Biodegradable materials suitable for the present disclosure include diamond, PLA, PGA, polypropylene sulfide, and PLGA polymers. Biocompatible but non-biodegradable materials may also be used in the carrier nanoparticles of the disclosure. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont), and nylons may be employed.

In some aspects, the nanoparticle disclosed herein comprises a biocompatible and/or a synthetic material including but not limited to, poly(lactic-co-glycolic acid) (PLGA), polylactic acid, polyglycolic acid, polycaprolactone, polylysine, polyglutamic acid, and any other suitable synthetic material or the like. In some aspects, the nanoparticle disclosed herein comprises poly(D, L-lactide-co-glycolide), or PLGA.

The nanoparticles disclosed herein can be prepared using any suitable methods available in the art. Non-limiting examples of methods that may be used for preparing nanoparticle disclosed herein include emulsion-solvent evaporation, emulsification-evaporation, nanoprecipitation or solvent displacement, solvent diffusion, and phase-inversion.

In some aspects, nanoparticles disclosed herein can be prepared using emulsion-solvent evaporation. Briefly, emulsion-solvent evaporation method comprises dissolving poly(D, L-lactide-co-glycolide) (PLGA) in a solvent such as for e.g., dichloromethane and adding dropwise to an emulsion stabilizer or a thickener, for e.g., polyvinyl-alcohol (PVA) solution. The resulting emulsion is then sonicated, for e.g., a probe sonicator (Fisherbrand) and added to a an emulsion stabilizer or a thickener solution, stirred for period of time to remove the solvent. Nanoparticles formed can be washed and collected by centrifugation.

The nanoparticle disclosed herein can have any suitable shape. For example, the present nanoparticle and/or its inner core can have a shape of sphere, circular, square, rectangle, triangle, circular disc, cube-like shape, cube, rectangular parallelepiped (cuboid), cone, cylinder, prism, pyramid, right-angled circular cylinder and other regular or irregular shape.

The nanoparticle disclosed herein can have any suitable size. In some aspects, the nanoparticle can have a hydrodynamic size of about 50-500 nm. In some aspects, the nanoparticles have a hydrodynamic size of about 50-100 nm, or about 100-150 nm, or about 150-200 nm, or about 200-250 nm, or about 250-300 nm, or about 300-350 nm, or about 350-400 nm, or about 400-450 nm or about 450-500 nm. Particle size can be affected by the polymer concentration; higher particles are formed from higher polymer concentrations. In some aspects, the nanoparticles disclosed herein can have a hydrodynamic size of about 200 nm. In some aspects, the nanoparticles disclosed herein can have a hydrodynamic size of about 250 nm. In some aspects, the nanoparticles disclosed herein can have a hydrodynamic size of about 280 nm. In some aspects, the nanoparticles disclosed herein can have a hydrodynamic size of about 290 nm.

In some aspects, current disclosure encompasses nanoparticles cloaked, coated, or encapsulated in lipid membrane to form a lipid nanoparticle complex. In some aspects, the lipid membrane can be any suitable biological membrane, for example a plasma membrane. In certain aspects, the nanoparticle cloaked, coated, or encapsulated with a naturally occurring cellular membrane and/or further comprises a synthetic membrane. In certain aspects, the plasma membrane is derived from a blood cell (e.g., red blood cell (RBC), white blood cell (WBC), or platelet). In other aspects, the plasma membrane is derived from an immune cell (e.g., macrophage, monocyte, B-cell, or T-cell). In particular aspects, the plasma membrane is from a blood cell. In some aspects, the plasma membrane is from a leucocyte or white blood cell. In some aspects, the plasma membrane is derived from a monocyte. In some aspects, the plasma membrane is derived from a mammalian monocyte. In some aspects, the plasma membrane is derived form a human monocyte.

In some aspects, the monocytes disclosed herein can be isolated from sources, non-limiting examples of which include bone marrow, blood sample, and peripheral blood mononuclear cell (PBMC). In some aspects, the monocytes disclosed herein can be isolated from bone marrow. In some aspects, the plasma membrane can be derived from the monocytes isolated from the subject that is need of a treatment, as disclosed herein. In certain aspects, the plasma membrane is isolated from a cell of the same species of the subject. In some aspects, monocytes isolated from a donor can be used. In such aspects, the monocytes may be collected from a single donor or multiple donors, In some aspects, the donor may be a matched donor. In such aspects, the donor may have the same blood type as that of the subject. In some aspects, the plasma membrane may be derived from a monocyte cell line, for e.g., THP-1, HL-60, SC cell line, or U937.

In some aspects, monocytes disclosed herein is a classical monocyte. In such aspects, monocytes comprise cells that express the markers: Ly6c, CD11b, and CCR2 (for e.g., Ly6c⁺, CD11b⁺, and CCR2⁺) and that do not express the marker: CD45R (for e.g., CD45R⁻)

In some aspects, the lipid membrane disclosed herein maintains natural structural integrity and activity of the plasma membrane. In some aspects, the structural integrity of the plasma membrane includes primary, secondary, tertiary or quaternary structure of the plasma membrane, and/or the activity of the cellular membrane which includes, but is not limited to, binding activity, receptor activity, signaling pathway activity, and any other activities a normal naturally occurring plasma membrane. In some aspects, the lipid bilayer structure and at least some of the associated membrane proteins embedded therewith in the disclosed plasma membrane are intact. In some aspects, the lipid nanoparticle complex or the cloaked nanoparticle mimics a cell surface. In some aspects, the lipid membrane mimics the plasma membrane of a monocyte.

In some aspects, the plasma membrane disclosed herein comprises at least one membrane protein. In some aspects, the membrane protein is an adhesion protein, a glycoprotein, or a type I transmembrane protein. In some aspects, the plasma membrane disclosed herein comprises an adhesion protein. In some aspects, the plasma membrane disclosed herein comprises a glycoprotein. In some aspects, the plasma membrane disclosed herein comprises a type I transmembrane protein. In some aspects, the plasma membrane disclosed herein comprises an adhesion protein, a glycoprotein, or a type I transmembrane protein, or any combination thereof.

Membranes may also comprise other agents that may or may not increase an activity of the lipid nanoparticle complex. In some aspects, functional groups such as antibodies and aptamers can be added to the outer surface of the membrane to enhance the activity or site targeting of the lipid nanoparticle complex.

In certain aspects, the lipid nanoparticle complex of the present disclosure is biocompatible and/or biodegradable. In some aspects, the nanoparticle of the lipid nanoparticle complex may comprise biodegradable and biocompatible polymer, for e.g. poly DL-lactide-co-glycolide (PLGA), polylactic acid (PLA), polyglycolic acid (PGA), polycaprolactone (PCL), polylysine, and/or polyglutamic acid. In some aspects, the lipid nanoparticle complex of the present disclosure comprises the plasma membrane derived from a monocyte and an inner core comprising PLGA.

In some aspects, the lipid nanoparticle complex or the cloaked nanoparticle can substantially lack constituents of the cell from which the lipid membrane is derived or its constituents. In certain aspects, the cloaked nanoparticle substantially lacks cytoplasm, nucleus and/or cellular organelles of the cell from which the plasma membrane is derived. In some aspects, the lipid membrane does not comprise a cytosolic protein of the cell from which the plasma membrane is derived. For example, the present lipid nanoparticle complex can lack, in terms of types and/or quantities, at least 10%, 20%, 30%, 40%, 50%, 60%, 70%, 80%, 90%, 95%, 96%, 97%, 98%, 99% or 100% the constituents of the cell from which the plasma membrane is derived. In some aspects, the lipid nanoparticle complex of the present disclosure comprises the plasma membrane derived from a monocyte and an inner core comprising PLGA, the lipid nanoparticle complex substantially lacking constituents of the cell from which the lipid membrane is derived. In some aspects, the lipid nanoparticle complex of the present disclosure comprises the plasma membrane derived from a monocyte and an inner core comprising PLGA, the lipid nanoparticle complex substantially lacking cytosolic proteins from which the lipid membrane is derived.

In some aspects, the lipid nanoparticle complex disclosed herein have a surface charge of about −15 to −20 mV. In some aspects, the lipid nanoparticle complex has a surface charge of about −15, or −16, or −17, or −18, or −19, or −20 mV. In some exemplary aspects, the complex has a surface charge of about −17.5 mV.

In some aspects, the lipid nanoparticle complex disclosed herein has increased stability. In some aspects, the present lipid nanoparticle complex is stable at −20 °C for at least 1, 2, 3, 4, 5, 6, 7, 8, 9, 10, 11, 12, 13, 14, or 15 days. In some aspects, the lipid nanoparticle disclosed herein is stable at least for 4 days at −20 °C. Stability of nanoparticles can be assessed for e.g., by assessing the particle aggregation, change in particle size and/or dispersity of the nanoparticles, using known methods in art.

In some aspects, the lipid nanoparticle complex disclosed herein has decreased toxicity. Toxicity of nanoparticles can be assessed for e.g., by assessing the hemolysis or organ toxicity via histological examination, or by quantifying weight loss in a subject. In some aspects, the present lipid nanoparticle complex can have at least 10%, 20%, 30%, 40%, 50%, 60%, 70%, 80%, 90%, 95%, 96%, 97%, 98%, 99% or 100% less toxicity, for e.g., 10%, 20%, 30%, 40%, 50%, 60%, 70%, 80%, 90%, 95%, 96%, 97%, 98%, 99% or 100% less hemolysis, or less than 80%, 70%, 60%, 50%, 40%, 30%, 20%, or 10% weight loss in a subject, compared to the weight at the time of or before administration of the lipid nanoparticle complex.

In some aspects, the lipid nanoparticle complex disclosed herein has enhanced uptake in endothelial cells compared to nanoparticles not cloaked, encapsulated or coated with a plasma membrane. In some aspects, the present lipid nanoparticle complex can have at least 10%, 20%, 30%, 40%, 50%, 60%, 70%, 80%, 90%, 95%, 96%, 97%, 98%, 99% or 100% enhanced uptake in endothelial cells compared to nanoparticles not cloaked, encapsulated or coated with a plasma membrane. In some aspects, lipid nanoparticle complex can have at least 10% enhanced uptake in endothelial cells compared to nanoparticles not cloaked, encapsulated or coated with a plasma membrane. In some aspects, lipid nanoparticle complex can have at least 40% enhanced uptake in endothelial cells compared to nanoparticles not cloaked, encapsulated or coated with a plasma membrane. Uptake of nanoparticles can be assessed using any known method in the art including cell staining, fluorescence microscopy and flow cytometry.

In some aspects, the lipid nanoparticle complex disclosed herein has enhanced uptake in inflamed endothelial cells compared to nanoparticles not cloaked, encapsulated or coated with a plasma membrane. In some aspects, the present lipid nanoparticle complex can have at least 10%, 20%, 30%, 40%, 50%, 60%, 70%, 80%, 90%, 95%, 96%, 97%, 98%, 99% or 100% enhanced uptake in inflamed endothelial cells compared to nanoparticles not cloaked, encapsulated or coated with a plasma membrane. In some aspects, lipid nanoparticle complex can have at least 10% enhanced uptake in inflamed endothelial cells compared to nanoparticles not cloaked, encapsulated or coated with a plasma membrane. In some aspects, lipid nanoparticle complex can have at least 40% enhanced uptake in inflamed endothelial cells compared to nanoparticles not cloaked, encapsulated or coated with a plasma membrane. Inflamed endothelial cells can be identified using known biomarkers including vascular cell adhesion protein-1 (VCAM-1) and uptake of nanoparticles can be assessed using any known method in the art including cell staining, fluorescence microscopy and flow cytometry.

In further aspects, the lipid nanoparticle complex disclosed herein has enhanced uptake in endothelial cells lining atherosclerotic vasculature compared to quiescent endothelial cells. In some aspects, the present lipid nanoparticle complex can have at least 10%, 20%, 30%, 40%, 50%, 60%, 70%, 80%, 90%, 95%, 96%, 97%, 98%, 99% or 100% enhanced uptake in endothelial cells lining atherosclerotic vasculature compared to quiescent endothelial cells. In some aspects, lipid nanoparticle complex can have at least 10% enhanced uptake in endothelial cells lining atherosclerotic vasculature compared to quiescent endothelial cells. In some aspects, lipid nanoparticle complex can have at least 40% enhanced uptake in endothelial cells lining atherosclerotic vasculature compared to quiescent endothelial cells. Inflamed endothelial cells can be identified using known biomarkers including vascular cell adhesion protein-1 (VCAM-1) and uptake of nanoparticles can be assessed using any known method in the art including cell staining, fluorescence microscopy and flow cytometry.

In some aspects the lipid nanoparticle complex disclosed herein has an increased half-life in the subject compared to nanoparticles not cloaked, encapsulated or coated with a plasma membrane. In some aspects, the lipid nanoparticle complex can have at least 10%, 20%, 30%, 40%, 50%, 60%, 70%, 80%, 90%, 95%, 96%, 97%, 98%, 99% or 100% increased half-life in the subject compared to nanoparticles not cloaked, encapsulated or coated with a plasma membrane. In some aspects, the lipid nanoparticle complex can have at least 10% increased half-life in the subject compared to nanoparticles not cloaked, encapsulated or coated with a plasma membrane. Half-lives of the nanoparticles can be assessed using amount of nanoparticles in blood circulation for a given amount of time, using methods known in the art.

In some aspects, the lipid nanoparticle complex disclosed herein further comprises a payload. In some aspects, the payload in the lipid nanoparticle complex disclosed herein comprises at least one therapeutic agent. In some aspects, any suitable therapeutic agent, the delivery of which would be more effective if incorporated into a lipid nanoparticle complex or a cloaked nanoparticle disclosed herein, can be used with this system. Non-limiting examples of suitable drugs include anticancer drugs, antineoplastic agent, a steroidal hormone, hormone, an anti-fungal drug, an anti-viral drug, an antibiotic, an opioid agonist, an opioid antagonist, a calcium channel blocker, an antiangiogenic drug, a diagnostic compound, a vitamin, interferon, macrophage activation factor, an interleukin, colony stimulating factor, tumor degenerating factor, epidermal growth factor, erythropoietin, tissue plasminogen activator, insulin, luteinizing hormone releasing hormone, an enzyme, a vaccine or an antibody.

In an exemplary aspect, the lipid nanoparticle complex disclosed herein comprises a therapeutic agent that inhibits the YAP/TAZ. In such aspects, therapeutic agent is an inhibitor. In some aspects, the inhibitor of the YAP/TAZ comprises a YAP/TAZ antagonist, e.g., an agent which inhibits the function or activity of YAP/TAZ. For example, the YAP/TAZ antagonist comprises a YAP/TAZ inhibitor. Alternatively, the YAP/TAZ antagonist includes an antagonist of a downstream YAP/TAZ target molecule. Suitable YAP/TAZ antagonists include an antibody or fragment thereof, a binding protein, a polypeptide, and any combination thereof. In some aspects, the YAP/TAZ antagonist comprises a nucleic acid molecule. Suitable nucleic acid molecules include double stranded ribonucleic acid (dsRNA), small hairpin RNA or short hairpin RNA (shRNA), small interfering RNA (siRNA), or antisense RNA, or any portion thereof. In another aspect, the YAP/TAZ antagonist comprises an optimized monoclonal anti-YAP antibody or anti-TAZ antibody. In another aspect, non-limiting examples of therapeutic agent that inhibits the YAP/TAZ include thiazovivin, cucurbitacin I, dasatinib, fluvastatin, pazopanib, statin drug (for e.g., mevastatin, pitavastatin, rosuvastatin, pentostatin (Nipent®), nystatin, lovastatin (Mevacor®), simvastatin (Zocor®), pravastatin (Pravachol®), fluvastatin (Lescol®), atorvastatin (Lipitor®), cerivastatin (Baycol®)), and verteporfin. In other aspects, the therapeutic agent that inhibits the YAZ/TAZ include β-adrenergic receptor agonists, Dobutamine, Latrunculin A, Latrunculin B, cytochalasin D, actin inhibitors, drugs that act on the cytoskeleton, Blebbistatitin, Botulinum toxin C3, and RHO kinase-targeting drugs (e.g., Y27632). In some aspects, the therapeutic agent is verteporfin. In some aspects, a therapeutically effective amount of the therapeutic agent can be incorporated into the nanoparticle.

In some aspects, the lipid nanoparticle complex disclosed herein comprises other therapeutic agents that can prevent, treat, or inhibit formation of atherosclerosis. In such aspects, the therapeutic agent is fibrates or fabric acid derivatives (for e.g., bezafibrate, ciprofibrate, fenofibrate, gemfibrozil, or clofibrate), biguanides (for e.g., metformin, phenformin, buformin), glitazones (for e.g., 5-((4-(2-(methyl-2-pyri-dinyl amino)ethoxy)-phenyl)methyl)-2,4-thiazolidinedione, troglitazone, pioglitazone, ciglitazone, WAY-120,744, englitazone, AD 5075, darglitazone, rosiglitazone), Sulfonylurea-based drugs (for e.g., glisoxepid, glyburide, acetohexamide, chlorpropamide, glibomuride, tolbutamide, tolazamide, glipizide, gliclazide, gliquidone, glyhexamide, phenbutamide, tolcyclamide), or any combination thereof.

In some aspects, the payload in the lipid nanoparticle complex disclosed herein comprises an imaging agent either in the core, or conjugated to the membrane enveloping the nanoparticle. Non-limiting examples of imaging agents can include ¹⁸F-FDG, or a radioisotope selected from a group consisting of ⁶⁸Ga, ^{99m}Tc, ¹¹¹In, ¹⁸F, ¹¹C, ¹²³I, ¹²⁴I and ¹³¹I.

In some aspects, the lipid nanoparticle complex of the present disclosure comprises the plasma membrane derived from a monocyte, an inner core comprising PLGA, and a payload comprising verteporfin.

In some aspect, the lipid nanoparticle complex disclosed herein enhances the accumulation of the payload in the plasma of the subject. In some aspects, the lipid nanoparticle complex disclosed herein enhances the accumulation of the payload in the plasma of the subject by at least 10%, 20%, 30%, 40%, 50%, 60%, 70%, 80%, 90%, 95%, 96%, 97%, 98%, 99% or 100% compared to nanoparticles not cloaked, encapsulated or coated with a plasma membrane. In some aspects, the lipid nanoparticle complex disclosed herein enhances the accumulation of the payload in the plasma of the subject by at least 10% enhanced compared to nanoparticles not cloaked, encapsulated or coated with a plasma membrane.

In some aspect, the lipid nanoparticle complex disclosed herein enhances the accumulation of the payload in the spleen of the subject. In some aspects, the lipid nanoparticle complex disclosed herein enhances the accumulation of the payload in the spleen of the subject by at least 10%, 20%, 30%, 40%, 50%, 60%, 70%, 80%, 90%, 95%, 96%, 97%, 98%, 99% or 100% compared to nanoparticles not cloaked, encapsulated or coated with a plasma membrane. In some aspects, the lipid nanoparticle complex disclosed herein enhances the accumulation of the payload in the spleen of the subject by at least 10% enhanced compared to nanoparticles not cloaked, encapsulated or coated with a plasma membrane.

In some aspect, the lipid nanoparticle complex disclosed herein decreases the accumulation of the payload in the liver of the subject. In some aspects, the lipid nanoparticle complex disclosed herein decreases the accumulation of the payload in the liver of the subject by at least 10%, 20%, 30%, 40%, 50%, 60%, 70%, 80%, 90%, 95%, 96%, 97%, 98%, 99% or 100% compared to nanoparticles not cloaked, encapsulated or coated with a plasma membrane. In some aspects, the lipid nanoparticle complex disclosed herein decreases the accumulation of the payload in the liver of the subject by at least 10% enhanced compared to nanoparticles not cloaked, encapsulated or coated with a plasma membrane.

### c. COMPOSITIONS

Further provided herein is a composition comprising the disclosed lipid nanoparticle complex. In some aspects, the composition comprises a lipid nanoparticle complex comprising a plasma membrane derived from a monocyte, an inner core comprising PLGA. In some aspects, the composition comprises a lipid nanoparticle complex comprising a plasma membrane derived from a monocyte, an inner core comprising PLGA and a payload comprising verteporfin. In some aspects, provided herein is a composition comprising an effective amount of disclosed lipid nanoparticle complex. In some aspects, the composition is a pharmaceutical composition.

In some aspects, the pharmaceutical compositions comprising the lipid nanoparticle complex disclosed herein comprises suitable carriers or excipients. Hereinafter, the phrases “physiologically acceptable carrier” and “pharmaceutically acceptable carrier” are interchangeably used to refer to a carrier or a diluent that does not cause significant irritation to an organism and does not abrogate the biological activity and properties of the administered compound. An adjuvant is included under these phrases.

In certain aspects, compositions disclosed herein may further comprise one or more pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some aspects, pharmaceutical compositions herein may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain aspects, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some aspects, any of the well-known techniques, carriers, and excipients may be used as suitable and/or as understood in the art.

In certain aspects, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, pharmaceutical compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some aspects, pharmaceutical compositions disclosed herein may comprise about 5% to about 99%, about 10%, about 95%, or about 15% to about 90% total amount of polymers as suspending agent(s) by total weight of the composition.

In certain aspects, pharmaceutical compositions disclosed herein may comprise a viscous formulation. In some aspects, viscosity of composition herein may be increased by the addition of one or more gelling or thickening agents. In some aspects, compositions disclosed herein may comprise one or more gelling or thickening agents in an amount to provide a sufficiently viscous formulation to remain on treated tissue. In some aspects, pharmaceutical compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some aspects, pharmaceutical compositions disclosed herein may comprise about 5% to about 99%, about 10%, about 95%, or about 15% to about 90% total amount of gelling or thickening agent(s) by total weight of the composition. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain aspects, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, or at least 50% total amount of one or more agents by total weight of the composition. In some aspects, pharmaceutical compositions disclosed herein may comprise about 5% to about 99%, about 10%, about 95%, or about 15% to about 90% total amount of one or more agents by total weight of the composition. In some aspects, one or more of these agents may be added to improve the performance, efficacy, safety, shelf-life and/or other property of the muscarinic antagonist composition of the present disclosure. In some aspects, additives may be biocompatible, without being harsh, abrasive, and/or allergenic.

In certain aspects, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In some aspects, pharmaceutical compositions disclosed herein may comprise at least 5%, at least 10%, at least 20%, at least 25%, at least 30%, at least 35%, at least 40%, at least 45%, at least 50% total amount of one or more acidifying agents by total weight of the composition. In some aspects, pharmaceutical compositions disclosed herein may comprise about 5% to about 99%, about 10%, about 95%, or about 15% to about 90% total amount of one or more acidifying agents by total weight of the composition.

In certain aspects, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more alkalizing agents by total weight of the composition. In some aspects, pharmaceutical compositions disclosed herein may comprise about 5% to about 99%, about 10%, about 95%, or about 15% to about 90% total amount of one or more alkalizing agents by total weight of the composition.

In certain aspects, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite and other materials known to one of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more antioxidants by total weight of the composition. In some aspects, pharmaceutical compositions disclosed herein may comprise about 5% to about 99%, about 10%, about 95%, or about 15% to about 90% total amount of one or more antioxidants by total weight of the composition.

In certain aspects, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more buffering agents by total weight of the composition. In some aspects, pharmaceutical compositions disclosed herein may comprise about 5% to about 99%, about 10%, about 95%, or about 15% to about 90% total amount of one or more buffering agents by total weight of the composition.

In some aspects, the amount of one or more buffering agents may depend on the desired pH level of a composition. In some aspects, pharmaceutical compositions disclosed herein may have a pH of about 6 to about 9. In some aspects, pharmaceutical compositions disclosed herein may have a pH greater than about 8, greater than about 7.5, greater than about 7, greater than about 6.5, or greater than about 6.

In certain aspects, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In some aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition. In some aspects, pharmaceutical compositions disclosed herein may comprise about 5% to about 99%, about 10%, about 95%, or about 15% to about 90% total amount of one or more preservatives by total weight of the composition.

In certain aspects, pharmaceutical compositions disclosed herein may comprise one or more surface-acting reagents or detergents. In some aspects, surface-acting reagents or detergents may be synthetic, natural, or semi-synthetic. In some aspects, compositions disclosed herein may comprise anionic detergents, cationic detergents, zwitterionic detergents, ampholytic detergents, amphoteric detergents, nonionic detergents having a steroid skeleton, or a combination thereof. In some aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition. In some aspects, pharmaceutical compositions disclosed herein may comprise about 5% to about 99%, about 10%, about 95%, or about 15% to about 90% total amount of one or more surface-acting reagents or detergents by total weight of the composition.

In certain aspects, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition. In some aspects, pharmaceutical compositions disclosed herein may comprise about 5% to about 99%, about 10%, about 95%, or about 15% to about 90% total amount of one or more stabilizers by total weight of the composition.

In some aspects, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some aspects, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some aspects, pharmaceutical compositions disclosed herein may comprise about 5% to about 99%, about 10%, about 95%, or about 15% to about 90% total amount of one or more tonicity modifiers by total weight of the composition.

In certain aspects, the present disclosure provides compositions formulated for one or more routes of administration. Suitable routes of administration may, for example, include intravenous, intracranial, intrathecal, subcutaneous, intranasal route, cranial, transmucosal, trans-nasal, transcranial, intracerebroventricular, intestinal, and/or parenteral delivery. In some aspects, compositions herein formulated can be formulated for parenteral delivery. In some aspects, compositions herein formulated can be formulated intramuscular, subcutaneous, intramedullary, intravenous, intraperitoneal, intracranial and/or intranasal injections.

In certain aspects, one may administer a composition disclosed herein in a local or systemic manner, for example, via local injection of the pharmaceutical composition directly into a tissue region of a patient. In some aspects, a pharmaceutical composition disclosed herein can be administered parenterally, e.g., by intravenous injection, intracerebroventricular injection, intra-cisterna magna injection, intra-parenchymal injection, or a combination thereof. In some aspects, a pharmaceutical composition disclosed herein can administered to subject as disclosed herein. In some aspects, a pharmaceutical composition disclosed herein can administered to human subject. In some aspects, a pharmaceutical composition disclosed herein can administered to a human subject via two or more administration routes. In some aspects, the combination of administration routes by be intracerebroventricular injection and intravenous injection; intrathecal injection and intravenous injection; intra-cisterna magna injection and intravenous injection; and/or intra-parenchymal injection and intravenous injection.

In certain aspects, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain aspects, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain aspects, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some aspects, compositions herein may be suspensions, solutions or emulsions in oily or aqueous vehicles, and/or may contain formulatory agents such as suspending, stabilizing and/or dispersing agents.

In some aspects, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some aspects, a therapeutically effective amount means an amount of active ingredients effective to prevent, slow, alleviate or ameliorate symptoms of a disorder or prolong the survival of the subject being treated.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

The optimal dosage to be administered will be readily determined by those skilled in the art and will vary on the condition being treated, the particular therapeutic agent and mode of administration. Other factors include the weight and condition of the subject.

A suitable, non-limiting example of a dosage of the disclosed composition comprising lipid nanoparticle complex according to the present disclosure may be from about 1 ng/kg to about 5000 mg/kg. In general, however, doses employed for adult human treatment typically may be in the range of 0.0001 mg/kg/day to 0.0010 mg/kg/day, 0.0010 mg/kg/day to 0.010 mg/kg/day, 0.010 mg/kg/day to 0.10 mg/kg/day, 0.10 mg/kg/day to 10.0 mg/kg/day, 10.00 mg/kg/day to about 200 mg/kg/day, 200 mg/kg/day to about 5000 mg/kg/day. For example, the dosage may be about 1 mg/kg/day to about 100 mg/kg/day, such as, e.g., 2-10 mg/kg/day, 10-50 mg/kg/day, or 50-100 mg/kg/day. The dosage can also be selected from about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, 2300 mg/kg, 2400 mg/kg, 2500 mg/kg, 2600 mg/kg, 2700 mg/kg, 2800 mg/kg, 2900 mg/kg, 3000 mg/kg, 3500 mg/kg, 4000 mg/kg, or 5000 mg/kg.

In some aspects, the lipid nanoparticle complex can be administered to the subject daily or more than once daily. In some aspects, the lipid nanoparticle complex can be administered every 2, 3, 4, 5, 6, 7, 14, or every 30 days. In some aspects, the lipid nanoparticle complex can be administered over a period ranging from about 1 day to about 1 year, from about 1 day to about 1 week, from about 3 days to about 1 month, from about 2 weeks to about 6 months, or from about 2 months to about 4 months. In some aspects, the lipid nanoparticle complex can also be administered over a period of about 1 day, about 7 days, about 30 days, about 60 days, about 120 days, or about 180 days or more. In some aspects, the lipid nanoparticle complex is administered over a period of about 57 weeks, about 148 weeks, about 208 weeks, indefinitely, or until resolution of the condition being treated.

### d. METHOD OF MAKING THE LIPID NANOPARTICLE COMPLEX

In some aspects, the current disclosure also encompasses methods of making and using the lipid nanoparticle complex described herein.

In various aspects, the current disclosure encompasses methods of preparing the nanoparticle lipid complexes described herein.

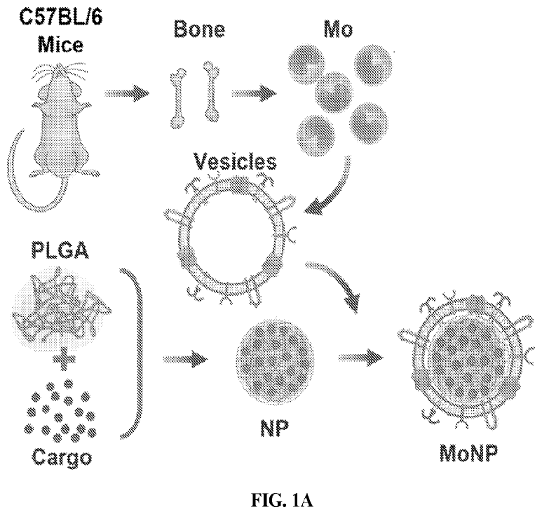

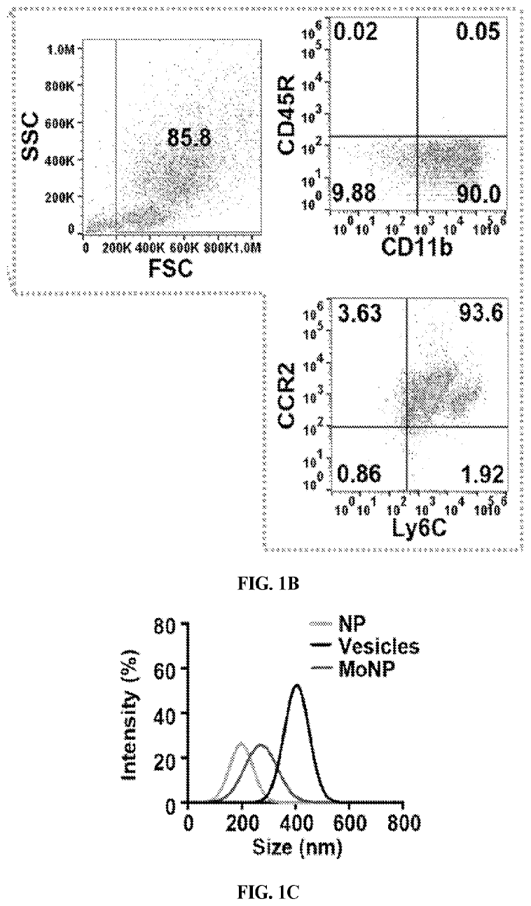

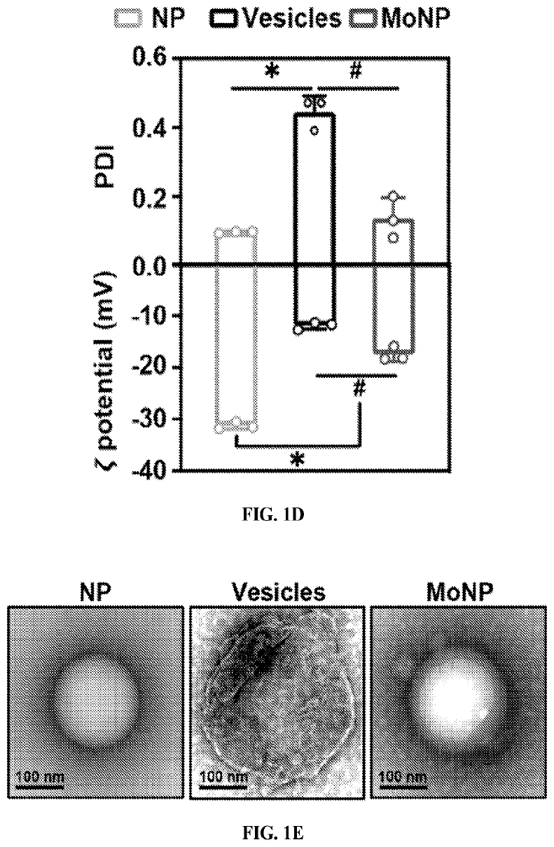

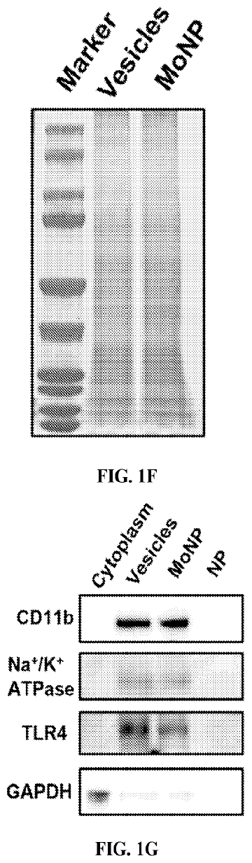

The nanoparticle lipid complexes may be prepared using a process as depicted in FIG. 1A. In general, the method comprises (a) forming a nanoparticle comprising an active pharmaceutical agent; (b) isolating a plasma membrane from a monocyte; and (c) contacting the plasma membrane of (b) with the nanoparticle of (a) to form the lipid nanoparticle complex. Details for each step are provided below.

As indicated, methods of preparing the complexes described herein first comprise preparing a nanoparticle. As used herein, a nanoparticle refers to a roughly spherical shaped unit that self-assembles under the appropriate conditions from an amphiphilic material so that the core is hydrophobic and the corona is hydrophilic. Nanoparticles may be prepared according to standard methods in the art.

The nanoparticles of the instant disclosure can be manufactured by any means commonly known in the art. Exemplary methods of manufacturing particles include, but are not limited to, microemulsion polymerization, interfacial polymerization, precipitation polymerization, emulsion evaporation, emulsion diffusion, solvent displacement, and salting out (Astete and Sabliov, J. Biomater. Sci. Polymer Edn., 17:247-289(2006)). Manipulation of the manufacturing process for PLGA particles can control particle properties (e.g. size, size distribution, zeta potential, morphology, hydrophobicity/hydrophilicity, polypeptide entrapment, etc). The size of the particle is influenced by a number of factors including, but not limited to, the concentration of PLGA, the solvent used in the manufacture of the particle, the nature of the organic phase, the surfactants used in manufacturing, the viscosity of the continuous and discontinuous phase, the nature of the solvent used, the temperature of the water used, sonication, evaporation rate, additives, shear stress, sterilization, and the nature of any encapsulated antigen or polypeptide.

In various aspects, the nanoparticle may be prepared using a method comprising PLGA emulsion which is described in Operti et al., (PLGA-based nanomedicines manufacturing: Technologies overview and challenges in industrial scale-up. International Journal of Pharmaceutics. Volume 605, 10 Aug. 2021, 120807) which is incorporated herein by reference in its entirety.

In further aspects, a plasma membrane is isolated from an immune cell (e.g., a monocyte). The isolation of said plasma membrane can occur by a variety of methods including agitation, introduction of a detergent, lysing, etc.

Finally, the plasma membrane isolated from the immune cell is contacted to the prepared nanoparticle. In some aspects, the plasma membrane is a monocyte. This may occur in a suitable composition or medium. In certain aspects, the method comprises exerting exogenous energy on the combination i.e., isolated plasma membrane and nanoparticle (for e.g., nanoparticle core). In certain aspects, the exogenous energy is a mechanical energy exerted by extrusion, In other aspects, the exogenous energy is an acoustic energy exerted by sonication. In yet other aspect, the exogenous energy is a thermal energy exerted by heating.

### e. METHODS OF TREATMENT

Methods for treating or preventing a disease or condition by administering the lipid nanoparticle complex according to the present disclosure is further provided. In such aspects, the method comprises administering the subject in need thereof lipid nanoparticle complex with or without a pharmaceutically acceptable carrier, adjuvant, or excipient. The present disclosure further provides the use of an effective amount of a composition comprising the lipid nanoparticle complex for the manufacture of a medicament for treating or preventing a disease or condition in subject in need.

In some aspects of the method, the lipid nanoparticle complex comprises a plasma membrane derived from a monocyte and an inner core comprising PLGA, the lipid nanoparticle complex substantially lacking constituents of the cell from which the lipid membrane is derived. In further aspects, the lipid nanoparticle complex comprises a plasma membrane derived from a monocyte and an inner core comprising PLGA, the lipid nanoparticle complex substantially lacking cytosolic proteins from which the lipid membrane is derived.

In some aspects, the compositions provided herein can be used in the treatment of atherosclerosis. Accordingly, in various aspects, a method of treating atherosclerosis in a subject in need thereof is provided, the method comprising administering an effective amount of a pharmaceutical composition comprising a nanoparticle lipid complex containing an active pharmaceutical agent to the subject in need thereof.

In some aspects of the method, the active pharmaceutical agent can be any therapeutic agent that can treat, prevent, or inhibit atherosclerosis. In some aspects, the therapeutic agent is an agent that inhibits the YAP/TAZ. In such aspects, therapeutic agent is an inhibitor. In some aspects, the inhibitor of the YAP/TAZ comprises a YAP/TAZ antagonist, e.g., an agent which inhibits the function or activity of YAP/TAZ. For example, the YAP/TAZ antagonist comprises a YAP/TAZ inhibitor. Alternatively, the YAP/TAZ antagonist includes an antagonist of a downstream YAP/TAZ target molecule. Suitable YAP/TAZ antagonists include an antibody or fragment thereof, a binding protein, a polypeptide, and any combination thereof. In some aspects, the YAP/TAZ antagonist comprises a nucleic acid molecule. Suitable nucleic acid molecules include double stranded ribonucleic acid (dsRNA), small hairpin RNA or short hairpin RNA (shRNA), small interfering RNA (siRNA), or antisense RNA, or any portion thereof. In another aspect, the YAP/TAZ antagonist comprises an optimized monoclonal anti-YAP antibody or anti-TAZ antibody. In another aspect, non-limiting examples of therapeutic agent that inhibits the YAP/TAZ include thiazovivin, cucurbitacin I, dasatinib, fluvastatin, pazopanib, statin drug (for e.g., mevastatin, pitavastatin, rosuvastatin, pentostatin (Nipent®), nystatin, lovastatin (Mevacor®), simvastatin (Zocor®), pravastatin (Pravachol®), fluvastatin (Lescol®), atorvastatin (Lipitor®), cerivastatin (Baycol®)), and verteporfin. In other aspects, the therapeutic agent that inhibits the YAZ/TAZ include β-adrenergic receptor agonists, Dobutamine, Latrunculin A, Latrunculin B, cytochalasin D, actin inhibitors, drugs that act on the cytoskeleton, Blebbistatitin, Botulinum toxin C3, and RHO kinase-targeting drugs (e.g., Y27632).

In some aspects of the method, the therapeutic agent is verteporfin. In such aspects, the method comprising administering an effective amount of a pharmaceutical composition comprising a nanoparticle lipid complex comprising verteporfin to the subject in need thereof. In such aspects, the lipid nanoparticle complex comprises a plasma membrane derived from a monocyte and an inner core comprising PLGA, and the lipid nanoparticle complex further comprising verteporfin, and substantially lacking cytosolic proteins from which the lipid membrane is derived.

In some aspects, the method of administering the lipid nanoparticle complex disclosed herein decreases arterial inflammation in the subject. In some aspects, administering the present lipid nanoparticle complex decrease by at least 10%, 20%, 30%, 40%, 50%, 60%, 70%, 80%, 90%, 95%, 96%, 97%, 98%, 99% or 100% arterial inflammation compared to compared to administration of nanoparticles not cloaked, encapsulated or coated with a plasma membrane, or administration with a therapeutic agent not encapsulated in a nanoparticle (for e.g., free verteporfin). In some aspects, lipid nanoparticle complex decreases arterial inflammation by at least 10% compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases arterial inflammation by at least 40% compared to administration of nanoparticles not cloaked, encapsulated or coated with a plasma membrane or administration with a therapeutic agent not encapsulated in a nanoparticle (for e.g., free verteporfin). Arterial inflammation can be assessed using known biomarkers including vascular cell adhesion protein-1 (VCAM-1), or Tumor necrosis factor alpha (TNFα).

In some aspects, the method of administering the lipid nanoparticle complex disclosed herein decreases atherosclerosis in the subject. In some aspects, administering the present lipid nanoparticle complex decrease by at least 10%, 20%, 30%, 40%, 50%, 60%, 70%, 80%, 90%, 95%, 96%, 97%, 98%, 99% or 100% atherosclerosis compared to compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases arterial inflammation by at least 10% compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases atherosclerosis by at least 70% compared to administration of nanoparticles not cloaked, encapsulated or coated with a plasma membrane or administration with a therapeutic agent not encapsulated in a nanoparticle (for e.g., free verteporfin). Atherosclerosis can be assessed by measuring atherosclerotic lesions or quantifying plaque size, by known methods in the art.

In some aspects, the method of administering the lipid nanoparticle complex disclosed herein decreases the size of atherosclerotic lesions in a carotid artery in the subject. In some aspects, administering the present lipid nanoparticle complex decrease by at least 10%, 20%, 30%, 40%, 50%, 60%, 70%, 80%, 90%, 95%, 96%, 97%, 98%, 99% or 100% the size of atherosclerotic lesions compared to compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the size of atherosclerotic lesions in a carotid artery by at least 10% compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the size of atherosclerotic lesions by at least 70% compared to administration of nanoparticles not cloaked, encapsulated or coated with a plasma membrane or administration with a therapeutic agent not encapsulated in a nanoparticle (for e.g., free verteporfin). Atherosclerotic lesions can be assessed by using histological methods in the art, such as for e.g., quantifying areas positive for Oil Red O or hematoxylin staining.

In some aspects, the method of administering the lipid nanoparticle complex disclosed herein decreases the plaque size in the subject. In some aspects, administering the present lipid nanoparticle complex decrease by at least 10%, 20%, 30%, 40%, 50%, 60%, 70%, 80%, 90%, 95%, 96%, 97%, 98%, 99% or 100% the plaque size compared to compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the plaque size by at least 10% compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the plaque size by at least 70% compared to administration of nanoparticles not cloaked, encapsulated or coated with a plasma membrane or administration with a therapeutic agent not encapsulated in a nanoparticle (for e.g., free verteporfin). Plaque size can be assessed by using histological methods in the art, such as for e.g., quantifying areas positive for Oil Red O or hematoxylin staining.

In some aspects, the method of administering the lipid nanoparticle complex disclosed herein decreases plaque development in the subject. In some aspects, administering the present lipid nanoparticle complex decrease by at least 10%, 20%, 30%, 40%, 50%, 60%, 70%, 80%, 90%, 95%, 96%, 97%, 98%, 99% or 100% plaque development compared to compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases plaque development by at least 10% compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases plaque development by at least 70% compared to administration of nanoparticles not cloaked, encapsulated or coated with a plasma membrane or administration with a therapeutic agent not encapsulated in a nanoparticle (for e.g., free verteporfin). Plaque development can be assessed by using histological methods in the art, such as for e.g., quantifying areas positive for Oil Red O or hematoxylin staining.

In some aspects, the method of administering the lipid nanoparticle complex disclosed herein decreases the luminal stenosis in the subject. In some aspects, luminal stenosis can be distal, middle, proximal stenosis, or a combination thereof. In some aspects, administering the present lipid nanoparticle complex decrease by at least 10%, 20%, 30%, 40%, 50%, 60%, 70%, 80%, 90%, 95%, 96%, 97%, 98%, 99% or 100% the luminal stenosis compared to compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the luminal stenosis by at least 10% compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the luminal stenosis by at least 70% compared to administration of nanoparticles not cloaked, encapsulated or coated with a plasma membrane or administration with a therapeutic agent not encapsulated in a nanoparticle (for e.g., free verteporfin). Luminal stenosis can be assessed by using histological methods in the art, such as for e.g., quantifying areas positive for Oil Red O or hematoxylin staining.

In some aspects, the method of administering the lipid nanoparticle complex disclosed herein decreases the luminal occlusion in the subject. In some aspects, administering the present lipid nanoparticle complex decrease by at least 10%, 20%, 30%, 40%, 50%, 60%, 70%, 80%, 90%, 95%, 96%, 97%, 98%, 99% or 100% the luminal occlusion compared to compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the luminal occlusion by at least 10% compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the luminal occlusion by at least 70% compared to administration of nanoparticles not cloaked, encapsulated or coated with a plasma membrane or administration with a therapeutic agent not encapsulated in a nanoparticle (for e.g., free verteporfin). Luminal occlusion can be assessed by using histological methods in the art, such as for e.g., quantifying areas positive for Oil Red O or hematoxylin staining.

In some aspects, the current disclosure further encompasses methods for targeted delivery of drugs using nanoparticles encapsulated with monocyte membrane and their use as biomimetic nanocarriers of drugs to achieve site-specific inhibition of YAP/TA-Z. In certain aspects, the method of targeted delivery can be used for treatment of atherosclerosis.

In some aspects, the method of administering the lipid nanoparticle complex disclosed herein can enhance the uptake of the lipid nanoparticle complex in endothelial cells of a subject compared to nanoparticles not cloaked, encapsulated or coated with a plasma membrane. In some aspects, the administration of the present lipid nanoparticle complex can have at least 10%, 20%, 30%, 40%, 50%, 60%, 70%, 80%, 90%, 95%, 96% 97%, 98%, 99% or 100% enhanced uptake in endothelial cells compared to nanoparticles not cloaked, encapsulated or coated with a plasma membrane. In some aspects, the administration of the lipid nanoparticle complex can have at least 10% enhanced uptake in endothelial cells of a subject compared to nanoparticles not cloaked, encapsulated or coated with a plasma membrane. In some aspects, the administration of the lipid nanoparticle complex can have at least 40% enhanced uptake in endothelial cells of a subject compared to nanoparticles not cloaked, encapsulated or coated with a plasma membrane. Uptake of nanoparticles can be assessed using any known method in the art including cell staining, fluorescence microscopy and flow cytometry.

In some aspects, the administration of the lipid nanoparticle complex disclosed herein has enhanced uptake in inflamed endothelial cells of a subject compared to nanoparticles not cloaked, encapsulated or coated with a plasma membrane. In some aspects, the administration of the lipid nanoparticle complex can have at least 10%, 20%, 30%, 40%, 50%, 60%, 70%, 80%, 90%, 95%, 96%, 97%, 98%, 99% or 100% enhanced uptake in inflamed endothelial cells of a subject compared to nanoparticles not cloaked, encapsulated or coated with a plasma membrane. In some aspects, the administration of the lipid nanoparticle complex can have at least 10% enhanced uptake in inflamed endothelial cells of a subject compared to nanoparticles not cloaked, encapsulated or coated with a plasma membrane. In some aspects, lipid nanoparticle complex can have at least 40% enhanced uptake in inflamed endothelial cells of a subject compared to nanoparticles not cloaked, encapsulated or coated with a plasma membrane. Inflamed endothelial cells can be identified using known biomarkers including vascular cell adhesion protein-1 (VCAM-1), or Tumor necrosis factor alpha (TNFα) and uptake of nanoparticles can be assessed using any known method in the art including cell staining, fluorescence microscopy and flow cytometry.

In further aspects, the administration of the lipid nanoparticle complex disclosed herein has enhanced uptake in endothelial cells lining atherosclerotic vasculature of a subject compared to quiescent endothelial cells. In some aspects, the administration of the present lipid nanoparticle complex can have at least 10%, 20%, 30%, 40%, 50%, 60%, 70%, 80%, 90%, 95%, 96%, 97%, 98%, 99% or 100% enhanced uptake in endothelial cells lining atherosclerotic vasculature of a subject compared to quiescent endothelial cells. In some aspects, the administration of the lipid nanoparticle complex can have at least 10% enhanced uptake in endothelial cells lining atherosclerotic vasculature in a subject compared to quiescent endothelial cells. In some aspects, the administration of the lipid nanoparticle complex can have at least 40% enhanced uptake in endothelial cells lining atherosclerotic vasculature in a subject compared to quiescent endothelial cells. Inflamed endothelial cells can be identified using known biomarkers including vascular cell adhesion protein-1 (VCAM-1), or Tumor necrosis factor alpha (TNFα) and uptake of nanoparticles can be assessed using any known method in the art including cell staining, fluorescence microscopy and flow cytometry.

In some aspects, the method of administering the lipid nanoparticle complex disclosed herein decreases the expression of YAP/TAZ in the subject. In some aspects, administering the present lipid nanoparticle complex decrease by at least 10%, 20%, 30%, 40%, 50%, 60%, 70%, 80%, 90%, 95%, 96%, 97%, 98%, 99% or 100% the expression of YAP/TAZ compared to compared to administration of nanoparticles not cloaked, encapsulated or coated with a plasma membrane or administration with a therapeutic agent not encapsulated in a nanoparticle (for e.g., free verteporfin). In some aspects, lipid nanoparticle complex decreases the expression of YAP/TAZ by at least 10% compared to administration of nanoparticles not cloaked, encapsulated or coated with a plasma membrane or administration with a therapeutic agent not encapsulated in a nanoparticle (for e.g., free verteporfin). In some aspects, the administration of lipid nanoparticle complex decreases the expression of YAP/TAZ by at least 70% compared to administration of nanoparticles not cloaked, encapsulated or coated with a plasma membrane or administration with a therapeutic agent not encapsulated in a nanoparticle (for e.g., free verteporfin). Expression of YAP/TAZ can be assessed using any known methods in the art, for e.g., RNA-seq, nanopore sequencing, nanostring, RT-PCR, NASBA, Fluorescence measurements or spectrophotometry.

In further aspects, the disclosure encompasses personalized method of treatment whereby the lipid nanoparticle complex is tailored to individual patients with reduced risk of immunogenicity by using their own plasma membranes for coating nanoparticles of the lipid nanoparticle complex. In such aspects, cells for e.g., monocytes are isolated from the individual. Cells such as monocytes can be isolated from a bone marrow sample provided by the individual. The plasma membrane then can be isolated using methods disclosed herein or using other methods known in the art. The isolated plasma membrane can be used to make lipid nanoparticle complex of the present disclosure, further comprising a therapeutic agent. The lipid nanoparticle complex can then be administered to the individual for treating a condition, such as for e.g., atherosclerosis.

In some aspects, the methods disclosed herein encompasses a method of treatment whereby the lipid nanoparticle complex comprises a plasma membrane which is isolated from the cells, for e.g., monocyte from a human Leucocyte Antigen (HLA) compatible donor. In such aspects, cells for e.g., monocytes are isolated from a donor individual or individuals who are HLA compatible. Cells such as monocytes can be isolated from a bone marrow sample provided by the donor individual, or pooled samples from multiple donor individuals who are HLA compatible. The plasma membrane then can be isolated using methods disclosed herein or using other methods known in the art. The isolated plasma membrane can be used to make lipid nanoparticle complex of the present disclosure, further comprising a therapeutic agent. The lipid nanoparticle complex can then be administered to the individual for treating a condition, such as for e.g., atherosclerosis.

In some aspects, the methods disclosed herein encompasses an allogenic method of treatment. In such aspects, cells for e.g., monocytes are isolated from a donor individual or individuals. Cells such as monocytes can be isolated from a bone marrow sample provided by the donor individual, or pooled samples from multiple donor individuals. The plasma membrane then can be isolated using methods disclosed herein or using other methods known in the art. The isolated plasma membrane can be used to make lipid nanoparticle complex of the present disclosure, further comprising a therapeutic agent. The lipid nanoparticle complex can then be administered to the individual for treating a condition, such as for e.g., atherosclerosis.

Accordingly, in various aspects, a method of targeted delivery of an active pharmaceutical agent is provided. Accordingly, in various aspects, a method of delivering a therapeutic to a subject in need thereof is provided, the method comprising administering an effective amount of a pharmaceutical composition comprising a nanoparticle lipid complex and the therapeutic to the subject in need thereof.

Accordingly, pharmaceutical compositions of the present disclosure comprise an effective amount of one or more nanoparticle-lipid complexes, optionally dissolved or dispersed in a pharmaceutically acceptable carrier.

The actual dosage amount of a composition of the present disclosure administered to an animal or a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Any suitable mode of administration can be used to administer the compositions provided herein in a subject in need thereof. Exemplary modes include, but are not limited to, intravenous injection. Other modes include, without limitation, intradermal, subcutaneous (s.c., s.q., sub-Q, Hypo), intramuscular (i.m.), intraperitoneal (i.p.), intraarterial, intramedullary, intracardiac, intra-articular (joint), intrasynovial (joint fluid area), intracranial, intraspinal, and intrathecal (spinal fluids). Any known device useful for parenteral injection of infusion of the formulations can be used to affect such administration.

In some aspects, the pharmaceutical compositions and methods provided herein may be administered in conjunction with other therapies for treating atherosclerosis. In some aspects, the administration may be done concurrently with other atherosclerosis therapies. In some aspects, the administration may be done before or after the administration of other atherosclerosis therapies. Non-limiting examples of other therapies include HMGCoA reductase inhibitors (statins), adjuvants, corticosteroids, anti-inflammatory compounds, analgesics, growth factors, antithrombotic agents, antiplatelet agents, fibrinolytic agents or thrombolytic agents.

In various aspects, a subject in need thereof can be having, suspected of having, or at risk of having at atherosclerosis. In various aspects, a subject in need thereof can have one or more symptoms or risk factors for atherosclerosis.

In various aspects, a suitable subject includes a human, a livestock animal, a companion animal, a lab animal, or a zoological animal. In one aspect, the subject may be a rodent, e.g., a mouse, a rat, a guinea pig, etc. In another aspect, the subject may be a livestock animal. Non-limiting examples of suitable livestock animals may include pigs, cows, horses, goats, sheep, llamas and alpacas. In yet another aspect, the subject may be a companion animal. Non-limiting examples of companion animals may include pets such as dogs, cats, rabbits, and birds. In yet another aspect, the subject may be a zoological animal. As used herein, a “zoological animal” refers to an animal that may be found in a zoo. Such animals may include non-human primates, large cats, wolves, and bears. In a specific aspect, the animal is a laboratory animal. Nonlimiting examples of a laboratory animal may include rodents, canines, felines, and non-human primates. In certain aspects, the animal is a rodent. Non-limiting examples of rodents may include mice, rats, guinea pigs, etc. In certain aspects, the subject is a human.

In an aspect, a disclosed method can comprise repeating one or more steps of a disclosed method and/or modifying one or more steps of a disclosed method (such as, for example, an administering step). In an aspect, a disclosed method of treatment can comprise modifying one or more of the administrations of the lipid nanoparticle complex. For example, modifying one or more of steps of the method of administration can comprise modifying or changing one or more features or aspects of one or more steps of a disclosed method. For example, in an aspect, a method can be altered by changing the amount of one or more of the disclosed lipid nanoparticle complex, disclosed vectors, disclosed pharmaceutical formulations, or a combination thereof administered to a subject, or by changing the frequency of administration of one or more of the disclosed lipid nanoparticle complex, disclosed vectors, disclosed pharmaceutical formulations, or a combination thereof to a subject, or by changing the duration of time one or more of the disclosed lipid nanoparticle complex.

In an aspect, a disclosed method of treatment in a subject in need thereof can further comprise monitoring the subject for adverse effects. In an aspect, in the absence of adverse effects, a disclosed method can further comprise continuing to treat the subject. In an aspect, in the presence of adverse effects, a disclosed method can further comprise modifying the treating step. Methods of monitoring a subject's well-being can include both subjective and objective criteria. Such methods are known to the skilled person.

### f. KITS

The present disclosure provides a kit comprising lipid nanoparticle complex for use with a method of the disclosure. The kit may comprise a composition comprising lipid nanoparticle complex, and instructions for administering the lipid nanoparticle complex to a subject in need thereof. The kit could further comprise other therapeutic agents, such as for e.g., an antithrombotic agent, that can be administered in combination with the lipid nanoparticle complex. In some aspects, the kit can further comprise a sterile, pharmaceutically acceptable carrier, buffer or other diluent. The kit provided herein generally include instructions for carrying out the methods. Instructions included in the kit may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The disclosed kit may have a single container that contains the disclosed lipid nanoparticle complex with or without any additional components, or they may have distinct containers for each desired agent. Where combined therapeutics are provided, a single solution may be pre-mixed, either in a molar equivalent combination, or with one component in excess of the other. Alternatively, lipid nanoparticle complex and other therapeutic agent components of the kit may be maintained separately within distinct container prior to the administration to a patient.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

The containers of the kit will generally include at least one vial, test tube, flask, bottle, syringe or other container means, into which the disclosed lipid nanoparticle complex, and any other desired agent, may be placed and, preferably, suitably aliquoted. Where separate components are included, the kit will also generally contain a second vial or other container into which these are placed, enabling the administration of separated designed doses. The kits may also comprise a second/third container means for containing a sterile, pharmaceutically acceptable buffer or other diluent.

The kit may also contain a means by which to administer the disclosed Ent-Testosterone to an animal or patient, e.g., one or more needles or syringes, or even an eye dropper, pipette, or other such like apparatus, from which the formulation may be injected into the animal or applied to a diseased area of the body. The kit of the present disclosure will also typically include a means for containing the vials, or such like, and other component, in close confinement for commercial sale, such as, e.g., injection or blow-molded plastic containers into which the desired vials and other apparatus are placed and retained.

Having described several aspects, it will be recognized by those skilled in the art that various modifications, alternative constructions, and equivalents may be used without departing from the spirit of the present disclosure. Additionally, a number of well-known processes and elements have not been described in order to avoid unnecessarily obscuring the present disclosure. Accordingly, this description should not be taken as limiting the scope of the present disclosure.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the disclosure is not entitled to antedate such disclosure by virtue of prior invention.

It is understood that the examples and aspects described herein are for illustrative purposes only and that various modifications or changes in light thereof will be suggested to persons skilled in the art and are to be included within the spirit and purview of this application and scope of the appended claims.

Unless specified, reagents employed in the examples are commercially available or can be prepared using commercially available instrumentation, methods, or reagents known in the art. The examples illustrate various aspects of the disclosure and practice of the methods of the disclosure. The examples are not intended to provide an exhaustive description of the many different aspects of the disclosure. Thus, although the disclosure has been described in some detail by way of illustration and example for purposes of clarity of understanding, those of ordinary skill in the art will realize readily that many changes and modifications can be made thereto without departing from the spirit or scope of the appended claims.

### Materials and Methods

### Isolation of Bone Marrow-Derived Mo and Mo Vesicles

Mouse Monocytes (Mo) were derived from bone marrow cells (BMC) isolated from the femurs, humerus, and tibiae of C57BL/6 mice. Briefly, BMC were flushed out from the bone and cultured in RPMI 1640 medium with 10% FBS, 1% penicillin-streptomycin, 1% L-glutamate, 1% sodium pyruvate, and 20 ng/mL M-CSF for 5 days. Mo were then isolated from the suspension of differentiated BMC using a MojoSort™ Mouse Monocyte Isolation Kit (BioLegend #480154). The expression of Mo surface markers was measured by flow cytometric analysis using fluorescently labeled antibodies against CD11b (BioLegend #101207), CD45R (BioLegend #103211), Ly6C (BioLegend #128021), and CCR2 (BioLegend #150627). To obtain Mo membrane vesicles, the plasma membrane fraction was separated using a Minute™ Plasma Membrane Protein Isolation and Cell Fractionation kit (Invent Biotechnologies).

NP were prepared using an emulsion-solvent evaporation method. Briefly, 10 mg of poly(D, L-lactide-co-glycolide) (PLGA; Resomer® RG 503H, Sigma Aldrich) was dissolved in 1 mL of dichloromethane (DCM, Sigma Aldrich) and added dropwise to 2% polyvinyl-alcohol (PVA, Acros Organics) solution. The resulting emulsion was sonicated with the probe sonicator (Fisherbrand) and added to a 0.5% PVA solution, then stirred for 3 hours to remove the solvent. NP were washed and collected by centrifugation. For the encapsulation of payloads, 400 g of DiD (Biotium) or 2 mg of VP (Tocris Bioscience) was dissolved in 1 mL of DCM containing PLGA. To prepare MoNP, the isolated Mo vesicles were added with NP at a membrane protein-to-NP weight ratio of 1:10 and mixed in an ultrasonic bath for 5 minutes. The hydrodynamic size, polydispersity index (PDI), and zeta potential of resulting particles were measured using dynamic light scattering (DLS) Zetasizer (Malvern). The morphology of MoNP and NP was examined after staining with 0.2% uranyl acetate using transmission electron microscopy (TEM) (Philips CM12) at ASU Eyring Materials Center. To determine the encapsulation efficiency (EE) and loading efficiency (LE), NP loaded with DiD or VP were lyophilized and then dissolved in dimethylsulfoxide (DMSO), followed by the measurement of fluorescent intensity using a plate reader (BioTek). EE and LE were calculated as follows:

$$EE ( \% ) = \frac{Output Mass ( VP or DiD )}{Input Mass ( VP or DiD )} \times 100 \%$$

$$LE ( \% ) = \frac{Output Mass ( VP or DiD )}{Input Mass ( PLGA ) + Input Mass ( VP or DiD )} \times 100 \%$$

### In Vitro Release of NP-DiD

The release of NP-DiD was assessed using the direct release method. Briefly, aliquots of NP-DiD were resuspended in three different solutions (saline pH 7, saline pH 7 containing 10% serum, and saline pH 6) and agitated at 100 rpm at 37 °C for predetermined time intervals ranging from 1 to 96 hours. Following incubation, the samples were centrifugated and dissolved in DMSO. The fluorescent intensity of the samples was then measured using a plate reader. The cumulative percentage of DiD released was calculated using the equation: Cumulative dye release (%)=(Read(0)−Read(t))/Read(0)×100), where Read(0) and Read(t) represent the amount of DiD loaded and the amount of DiD released at time t, respectively.

### Cellular Uptake and Intracellular Fate of MoNP

Human umbilical cord vein EC (ATCC), Mo, and macrophages derived from mouse BMC were maintained in their respective growth medium under standard cell culture conditions (37 °C, 5% CO2, 100% humidity). For the cellular uptake assays, MoNP-DiD or NP-DiD were incubated with untreated EC, TNFα-pretreated EC, TNFα-/anti-VCAM1-pretreated EC, Mo, and macrophages for 30 minutes and then replaced with fresh medium. For the shear stress (SS) experiment, MoNP-DiD were added to the circulating medium in a parallel plate flow system and allowed to interact with EC exposed to high or low SS (12 or 1 dyne/cm²) for 2 hours. The intracellular DiD signal was quantified using fluorescent imaging and flow cytometric analyses. For the colocalization study, MoNP- or NP-treated EC were stained with LysoTracker (Cell Signaling) and DAPI and observed using the confocal microscope (Leica SP8) at ASU Advanced Light Microscopy Facilities. Colocalization of MoNP-DiD or NP-DiD with lysosomes was determined using the JACoP plugin in ImageJ on randomly selected cells and calculation of Pearson's correlation coefficients.

### Biocompatibility Testing

To assess cytotoxicity of MoNP, EC were incubated with MoNP, NP, or PBS. The number of live EC was quantified on day 1, 2, and 3 post-seeding. Hemolysis induced by MoNP or NP was evaluated in vitro using the direct contact method. Briefly, mouse blood was diluted with saline and mixed with MoNP, NP, or deionized water as a control. The samples were incubated at 37 °C for 1 hour and visually inspected after low-speed centrifugation. The supernatant absorbance was measured using a plate reader at 540 nm. For in vivo toxicity, apolipoprotein E-deficient (ApoE−/−) mice were intravenously administered with MoNP or saline every 72 hours for a total of three injections. On day 9, the organs were harvested, sectioned, and stained with hematoxylin and eosin for histopathological analysis.

### SDS-PAGE and Western Blot Analysis

Equal amounts of protein were separated by sodium dodecyl sulfate-polyacrylamide gel electrophoresis (SDS-PAGE) and stained with a Gel-FAST Gel Staining/Destaining kit (BioVision) for visualization. For Western blot analysis, the proteins were transferred to nitrocellulose membranes after electrophoresis, followed by blocking with 5% BSA and incubation with primary and secondary antibodies. The following antibodies were used in this study at a dilution of 1:1000: CD11b (Cell Signaling #17800), Na+/K+ ATPase (Cell Signaling #3010), VCAM1 (Cell Signaling #13622), ICAM1 (Cell Signaling #4915), and GAPDH (Cell Signaling #2118), TLR4 (BioLegend #145401), Histone 3 (BioLegend #819411), YAP/TAZ (Santa Cruz #sc-101199), and CTGF (Novus Biologicals #NB100-724SS). The blots were developed using a chemiluminescence kit (Pierce) and imaged using the Analytik Jena bioimaging system. The band intensity was quantified using ImageJ. Uncropped Western blot images are displayed in Supplementary Data.

### RNA Extraction and Analysis

Total RNA was extracted using TRIzol reagent (Invitrogen) according to the manufacturer's protocols. Subsequently, mRNA was reverse-transcribed to cDNA by using oligo-dT primer (Promega) and M-MLV reverse transcriptase (Promega). Quantitative real-time PCR (qRT-PCR) was performed on a QuantStudio 3 Real-Time PCR System (Applied Biosystems). Relative gene expression was determined by the 2^{-ΔΔCT} method, and the primer sets used for qRT-PCR are listed in Table 1. For RNA-sequencing (RNA-seq) transcriptome profiling, the RNA library and transcriptome sequencing was conducted by Novogene. The sequence read pairs were aligned to the human genome (hg38) using STAR, and the number of reads for each gene was counted using StringTie. DESeq2 was used to identify differentially expressed genes (DEGs), and genes with the sum of counts less than 100 across all samples were removed before the analysis. The heatmap was generated by pheatmap (version 1.0.12) with a row z-score value. Genes with a multiple hypotheses-corrected P value of <0.05 and |log₂FoldChange|≥0.75 were considered DEGs. DEGs were subjected to ClusterProfiler, including the Kyoto Encyclopedia of Genes and Genomes (KEGG) pathway enrichment and Gene Ontology (GO) analysis. The RNA-seq data are available in GEO DataSets with the accession number GSE229918.

TABLE 1
List of primers for qRT-PCR

| Gene | Species | Sequence (5′→3′) |
|---|---|---|
| VCAM1 | human | F: ggcagagtacgcaaacactt |
| | | (SEQ ID NO: 1) |
| | | R: acaggattttcggagcagga |
| | | (SEQ ID NO: 2) |
| ICAM1 | human | F: cttgagggcacctacctctg |
| | | (SEQ ID NO: 3) |
| | | R: cattatgactgcggctgcta |
| | | (SEQ ID NO: 4) |
| GAPDH | human | F: gcaccaccaactgcttagc |
| | | (SEQ ID NO: 5) |
| | | R: atgatgttctggagagcccc |
| | | (SEQ ID NO: 6) |
| CTGF | human | F: ggcccagacccaactatgat |
| | | (SEQ ID NO: 7) |
| | | R: aggaggcgttgtcattggta |
| | | (SEQ ID NO: 8) |

F: forward; R: reverse

### Mo Adhesion Assay

THP-1 cells were labeled with CellBrite® Cytoplasmic Membrane Dye (Biotium) and resuspended to the concentration of 5×105 cells/mL. The labeled THP-1 cells were then added to TNFα-stimulated or control EC monolayers pretreated with MoNP-VP or MoNP. After incubation, unattached THP-1 cells were washed out with fresh medium. Fluorescent images showing Mo attachment to EC monolayers were taken under Lionheart FX Automated Microscope, and the number of adherent THP-1 cells was quantified using ImageJ.

### Animal Experiments

All animal experiments performed in this study have been approved by the Institutional Animal Care and Use Committee (IACUC) of Arizona State University, USA. Male ApoE^{-/-} mice (8-12 weeks old), purchased from The Jackson Laboratory, were subjected to the partial ligation (PL) procedure to induce inflammation and carotid atherosclerosis in the left carotid artery (LCA). All mice were fed a high-fat diet (Envigo #TD.88137) for 1 week prior to the PL procedure and continued to be fed the same diet until the time of euthanasia. The PL was conducted by ligating the left external CA, left internal CA, and left occipital artery while maintaining the left superior thyroid artery intact. For biodistribution analysis, MoNP-DiD was administered via retro-orbital injection to mice that underwent the PL 1 week prior. The arterial tissue (from carotid bifurcation to abdominal aorta) and organs were harvested 4 hours after the injection, and DiD intensity was quantified for each of the three individual zones within the arterial segments using fluorescent imaging. Subsequently, the left common CA was embedded in Tissue-Tek O.C.T. compound, cryosectioned, and stained with a CD31 antibody (R&D Systems #AF3628) and DAPI. For organ assessment, various tissues (kidney, lung, heart, gut, liver, and spleen) and plasma were homogenized, and the fluorescent intensity in homogenized tissues was measured. For the short-term experiment, MoNP-VP at a dose of 2 mg/kg was administered intravenously three times a week to assess the effect on PL-induced inflammatory response. Arterial tissues were harvested 24 hours after the 3rd injection of MoNP-VP for Western blot and immunofluorescence analysis using antibodies against YAP/TAZ, CTGF, VCAM1, and CD68 (BioLegend #137001). To evaluate the long-term effect on plaque development, the mice underwent PL and were then injected with MoNP-VP, free VP, MoNP, and saline via intravenous injection every 3 days for a total of 6 injections. Four weeks after PL, arterial tissues were harvested for lesion assessment. The LCA was cryosectioned and stained with Oil Red O and hematoxylin to evaluate the plaque size and luminal stenosis. Major organs were harvested, cryosectioned, and stained with hematoxylin and eosin for histopathological examination. Serum samples isolated from the mice were analyzed to determine their lipid profile and metabolic panel. These analyses were carried out by Protatek Reference Laboratory (Mesa, AZ).

### Statistical Analysis

All graphical data are presented as mean±standard deviation (SD), and statistical analysis from at least three biological repeat experiments were performed using GraphPad Prism 8.0. For comparisons between two groups, a two-tailed unpaired Student's t-test was used. One-way ANOVA was performed for the comparison of multiple groups, and statistical significance among multiple groups was determined by Tukey's multiple comparisons test.

### Example 1: Formulation and Characterization of Monocyte Membrane-Cloaked Nanoparticle (MoNP)

To achieve active targeting to activated and inflamed endothelium, the biomimetic MoNP using the plasma membrane of mouse Mo and PLGA NP was prepared (FIG. 1A). Briefly, NP were prepared using an oil-in-water-emulsion method, after which they were cloaked with the membrane vesicles from mouse classical Mo (Ly6c⁺/CD11b⁺/CCR2⁺/CD45R⁻) isolated from the suspension of BMC differentiated in the medium containing M-CSF (FIG. 1B). The resulting MoNP were then subjected to physicochemical characterization using DLS, TEM, SDS-PAGE, and Western blot analyses. The DLS data showed that MoNP were highly uniform in size, with an average hydrodynamic size of 284.74 nm, while Mo vesicles and uncoated NP are 430.26 nm and 216.36 nm, respectively (FIG. 1C). MoNP had a mean surface charge of −17.45 mV, which was much closer to that of Mo vesicles than NP (i.e., −11.88 mV for Mo vesicles and −31.26 mV for NP), indicating successful membrane cloaking (FIG. 1D). The TEM analysis confirmed that Mo membrane was fused onto NP to form a core-shell nanostructure with a final diameter slightly over 200 nm (FIG. 1E). Furthermore, our SDS-PAGE and Western blot results demonstrated that CD11b, Na⁺/K⁺ ATPase, and TLR4, but not GAPDH, were decorated on the surface of MoNP, verifying that Mo membrane proteins, rather than their intracellular components, were present on MoNP (FIG. 1F-1G).

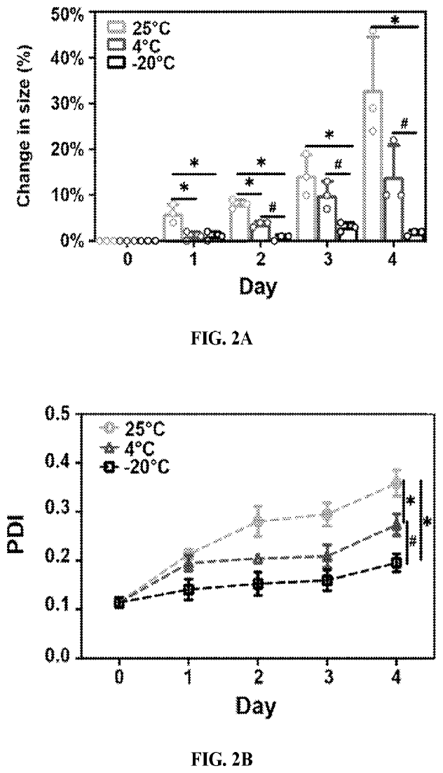

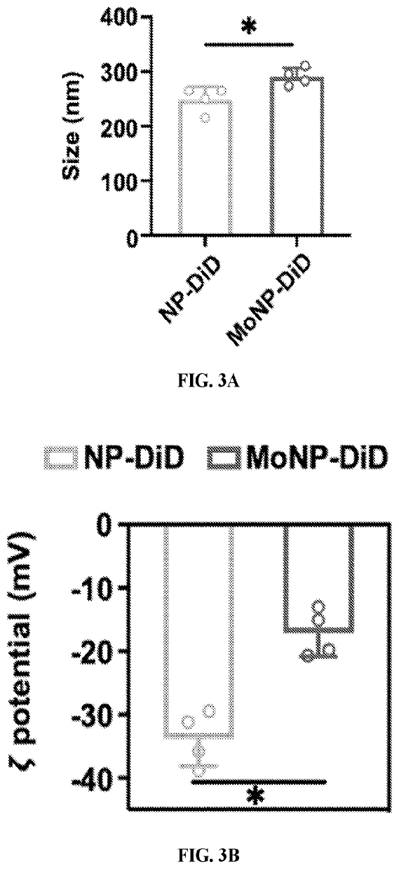

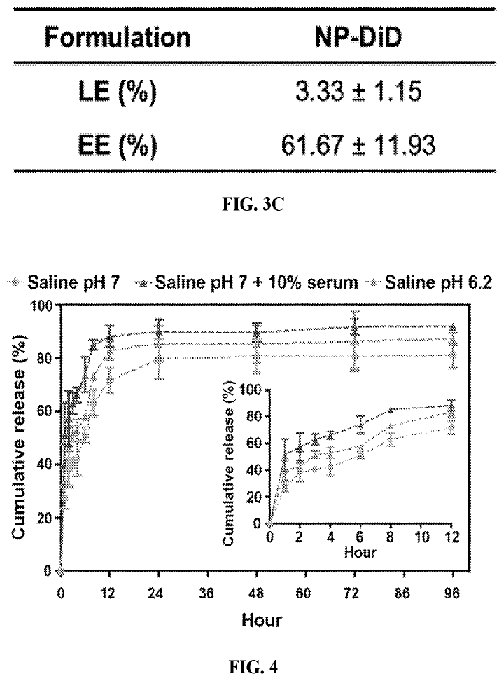

The stability of MoNP was then investigated by storing aliquots at different temperatures (25 °C, 4 °C, or −20 °C) for 4 days. The findings showed that storing MoNP at −20 °C significantly reduced particle aggregation and helped maintain monodispersity even after the freezing-thawing process (FIG. 2A-2B). To assess the drug loading capacity and release profile, the MoNP was formulated with the fluorescent dye DiD as a payload. Approximately 61% of the input DiD could be encapsulated into MoNP, attaining a LE of 3.3%, and the final hydrodynamic size remained stable at approximately 290 nm (FIG. 3A-3C). Additionally, when incubated with a saline solution containing 10% serum, approximately 90% of the payload could be released from NP within 24 hours (FIG. 4).

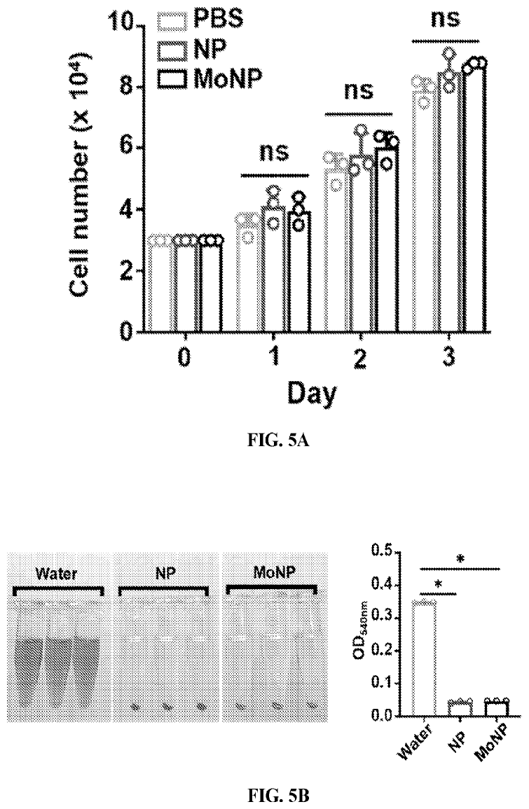

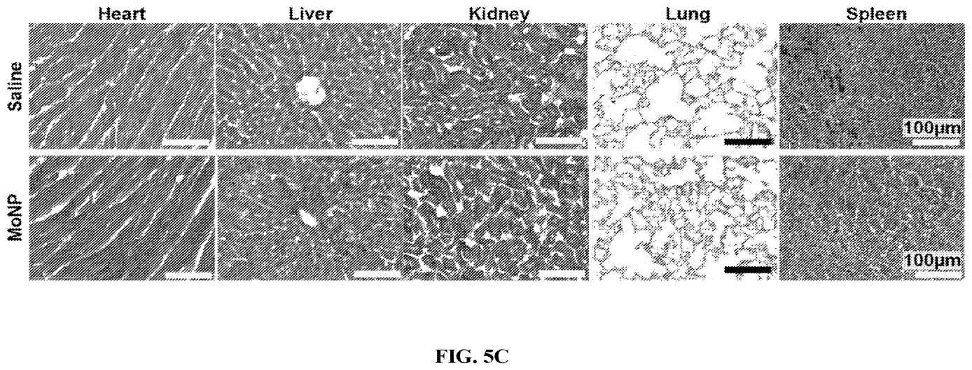

The biocompatibility of MoNP was further assessed by evaluating their in vitro and in vivo toxicity. First, EC was incubated with MoNP and their growth was monitored, and it was observed that the presence of MoNP did not affect cell proliferation (FIG. 5A). Mouse whole blood was also incubated with MoNP and was found that MoNP did not cause apparent hemolysis (FIG. 5B). Moreover, MoNP was administered intravenously to mice and organ toxicity was evaluated via histological examination. MoNP did not induce any noticeable histopathological changes in major organs, including the heart, liver, kidney, lung, and spleen (FIG. 5C). These results indicated that MoNP was a highly biocompatible vehicle for drug delivery, with no significant toxic effects observed in cellular or animal models.

### Example 2: MoNP Facilitate Selective Uptake by Inflamed EC Rather than Phagocytes

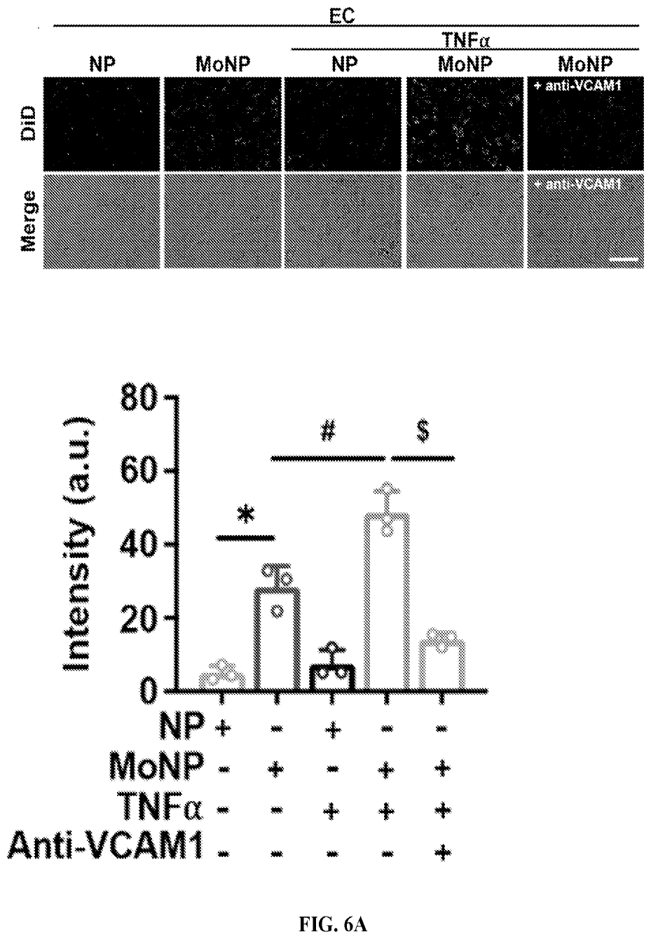

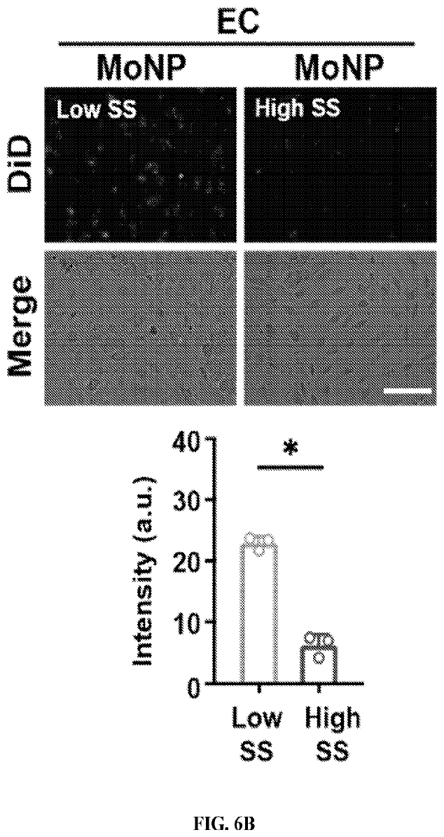

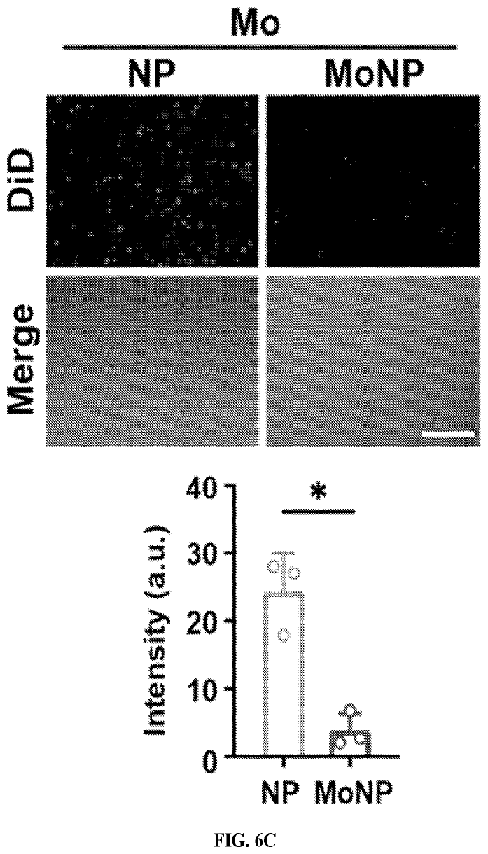

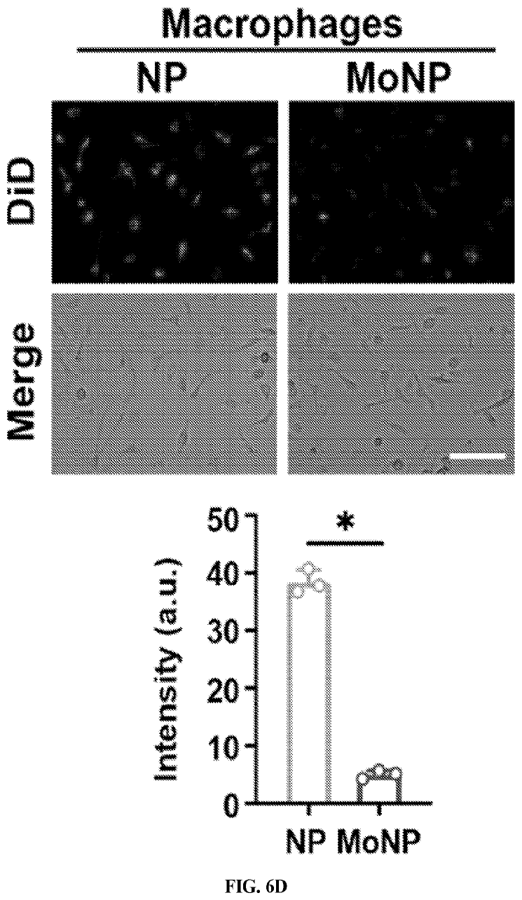

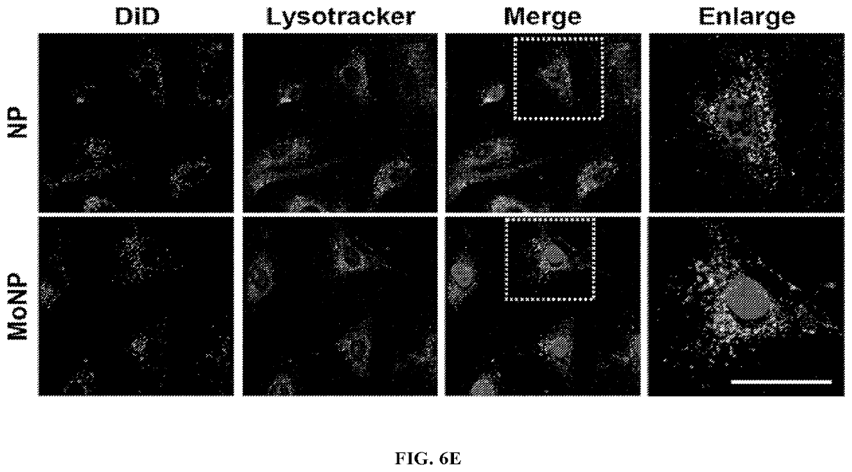

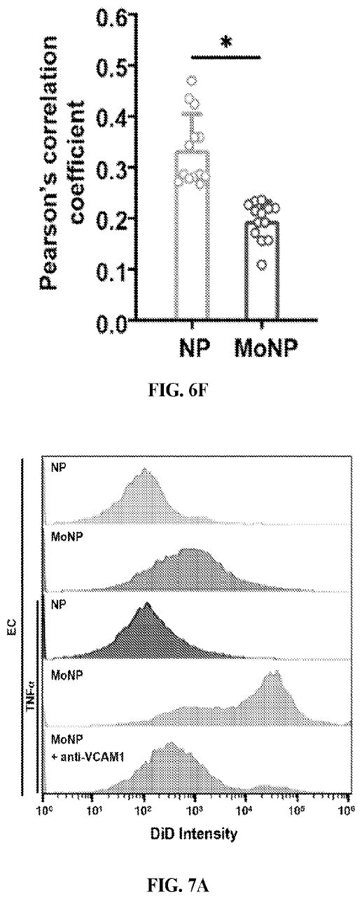

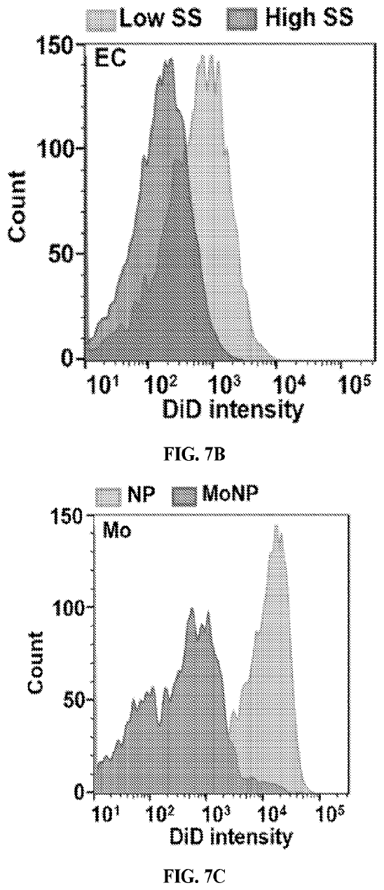

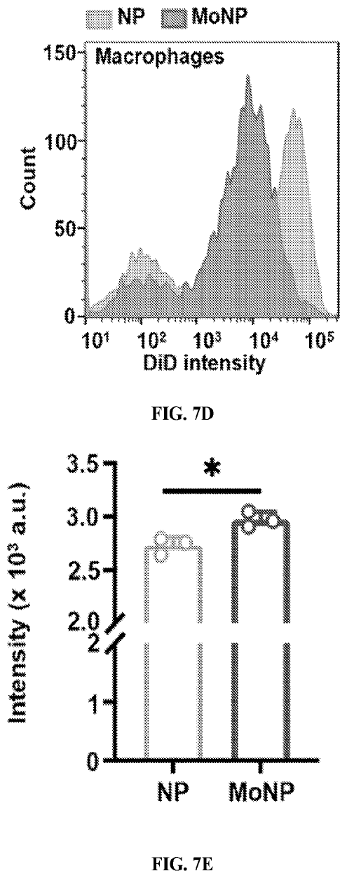

Next, to investigate the impact of MoNP on cellular uptake, EC and Mo, respectively, were incubated with MoNP-DiD or uncoated NP-DiD, followed by analyses of the intracellular DiD signal using fluorescence microscopy and flow cytometry. MoNP-DiD treatment, compared to uncoated NP-DiD, significantly increased the number of DiD-positive EC, especially in those pre-treated with proinflammatory cytokine TNFα. Notably, pretreatment of an antibody against VCAM1 abolished the increased uptake of MoNP-DiD in inflamed EC (FIG. 6A and FIG. 7A). The uptake of MoNP-DiD by EC exposed to different magnitudes of SS was also tested. Using a parallel plate flow system, it was observed that more MoNP accumulated in EC under low SS, which is commonly observed in atheroprone regions and found to promote EC inflammation, than those under high SS (FIG. 6B and FIG. 7B). In contrast to the findings in EC, mouse phagocytes, including Mo and macrophages, internalized more uncoated particles than those enclosed in Mo membrane (FIGS. 6C-6D, and FIGS. 7C-7D). Additionally, mouse whole blood was incubated with MoNP-DiD or NP-DiD and found a stronger DiD signal in the plasma of the MoNP-DiD group than that of NP-DiD (FIG. 7E). These results suggested that Mo membrane cloaking enhanced the interaction between MoNP and inflamed endothelium in a VCAM1-dependent manner and reduced uptake by immune phagocytes. To investigate the intracellular fate of MoNP, EC was incubated with MoNP-DiD or NP-DiD and then the lysosomes stained using LysoTracker Green. Compared to NP, MoNP showed a lower colocalization with lysosomes than the NP group, indicating that Mo membrane coating could facilitate the escape of resulting particles from lysosomes after internalization and potentially extend their therapeutic effects (FIG. 6E-6F).

These findings indicated that the monocyte membrane cloaking enhanced the interaction between MoNP and inflamed endothelium in a VCAM1-dependent manner and reduced the phagocytic clearance.

### Example 3: MoNP Preferentially Accumulate in Intima of Arterial Regions Prone to Atherosclerosis

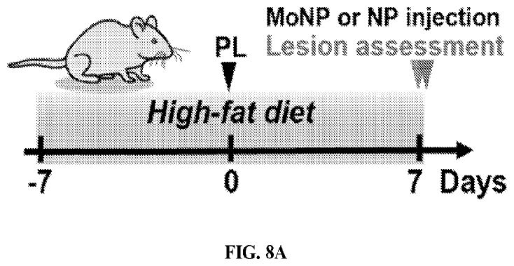

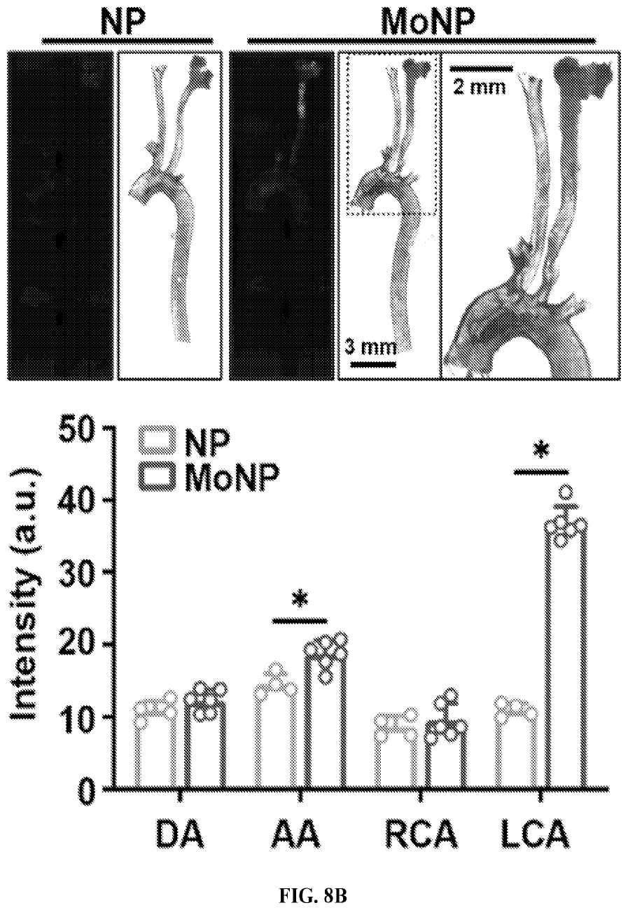

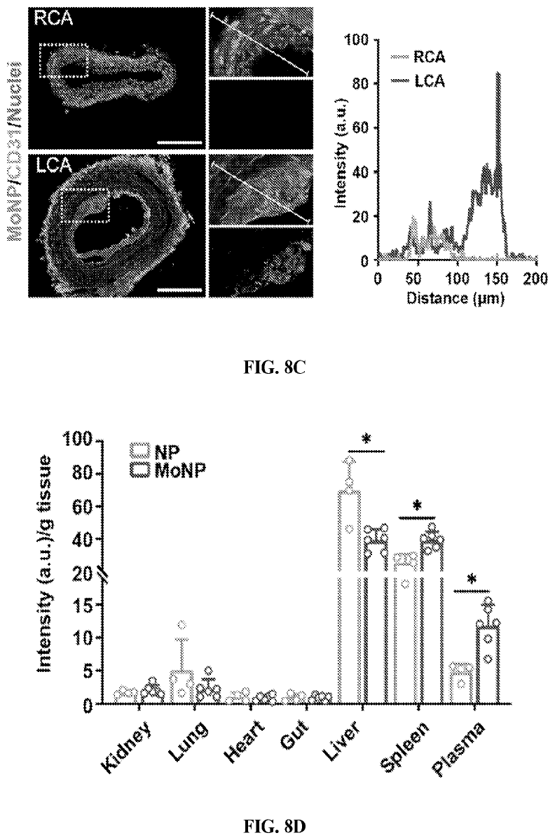

Next, to determine whether MoNP could achieve active targeting of inflamed EC lining atherosclerotic vasculature in vivo, the PL procedure was performed in ApoE^{-/-} mice. PL creates flow disturbance in the LCA and is a well-established approach to induce endothelial activation/dysfunction and accelerate the development of carotid atherosclerosis. Seven days after PL, MoNP-DiD or NP-DiD was intravenously administered to the mice. Four hours post-injection, arterial tissues were harvested from descending aorta to carotid bifurcations and major organs to determine the biodistribution of administered particles (FIG. 8A). Fluorescent imaging of whole-mount arterial tissues revealed a strong DiD signal in the partially ligated LCA, followed by aortic arches, in mice receiving MoNP-DiD but not in those receiving NP-DiD. Additionally, only background signal was detected in intact regions of the vasculature, such as descending aorta and right carotid artery (RCA), of these mice (FIG. 8B). Imaging of tissue sections confirmed these observations by showing that DiD signal was primarily delivered to the thickened intima of the LCA, with little-to-no fluorescence detected in the normal intima of the RCA (FIG. 8C). These results provided strong evidence that Mo membrane cloaking enhanced the interaction of the resulting particles with inflamed endothelium but not quiescent EC of the vasculature. Moreover, the biodistribution analysis showed that compared to the uncoated NP, MoNP-mediated delivery increased the fluorescent payload in the plasma and spleen and decreased its accumulation in the liver (FIG. 8D). These findings indicated that while MoNP and NP have similar clearance routes through the liver and spleen, MoNP have extended residence time in circulation. Importantly, MoNP enabled site-specific delivery of the payload to the inflamed endothelium displayed over plaque tissues, highlighting the potential of MoNP as a targeted delivery approach for atherosclerosis.

### Example 4: MoNP-VP Treatment Alleviates the TNFα-Induced Inflammatory Responses in EC

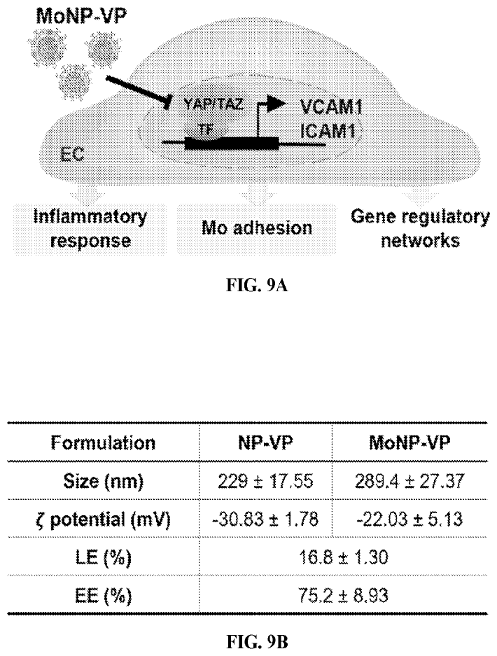

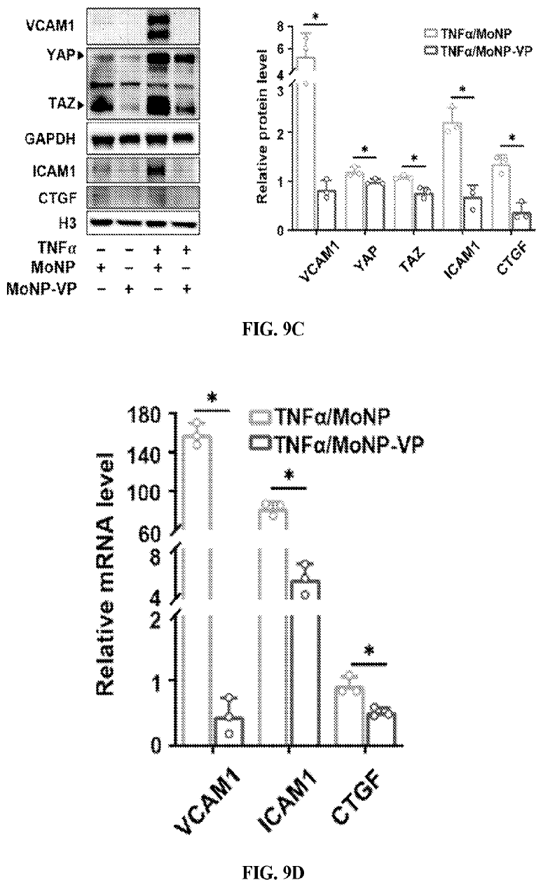

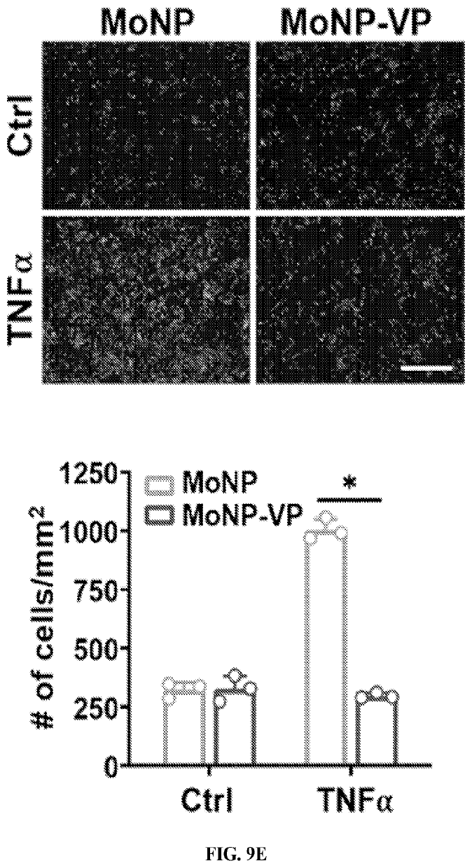

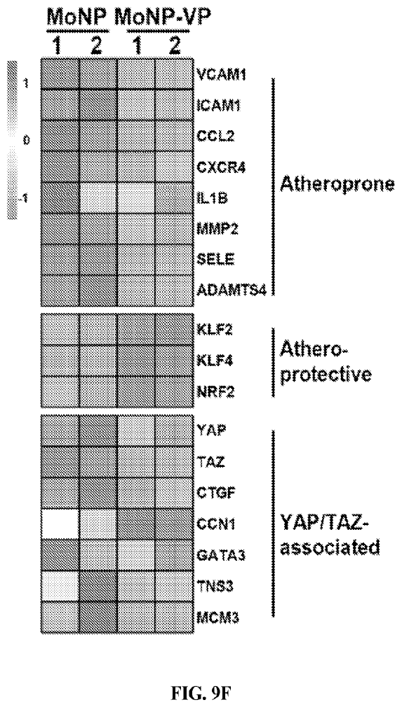

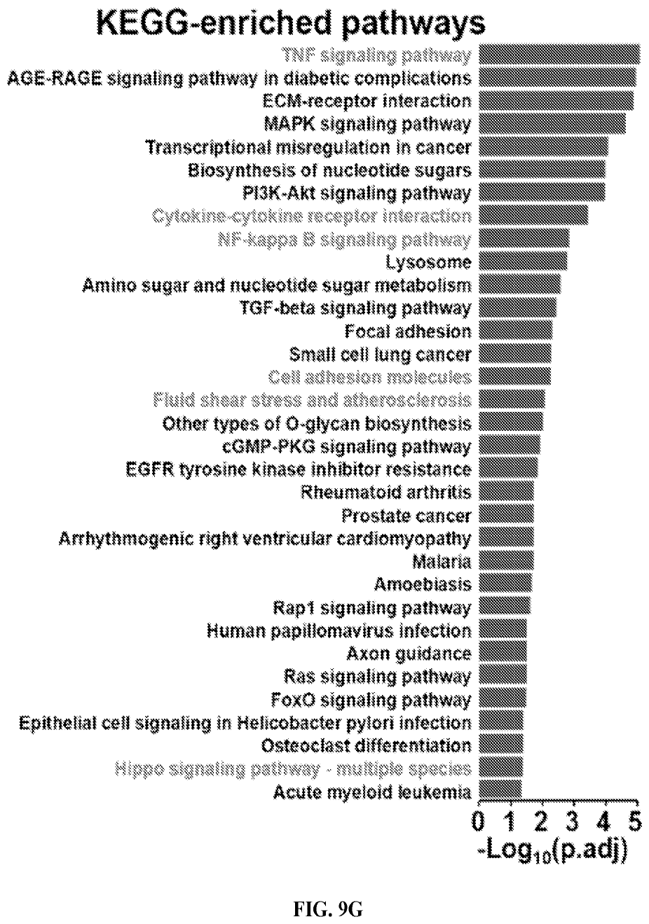

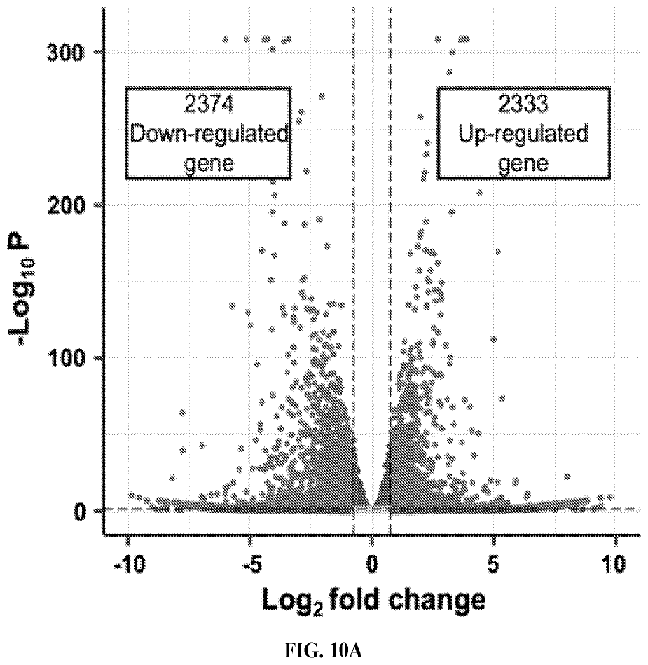

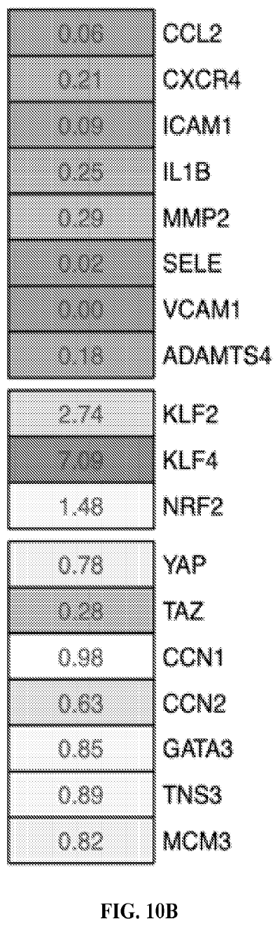

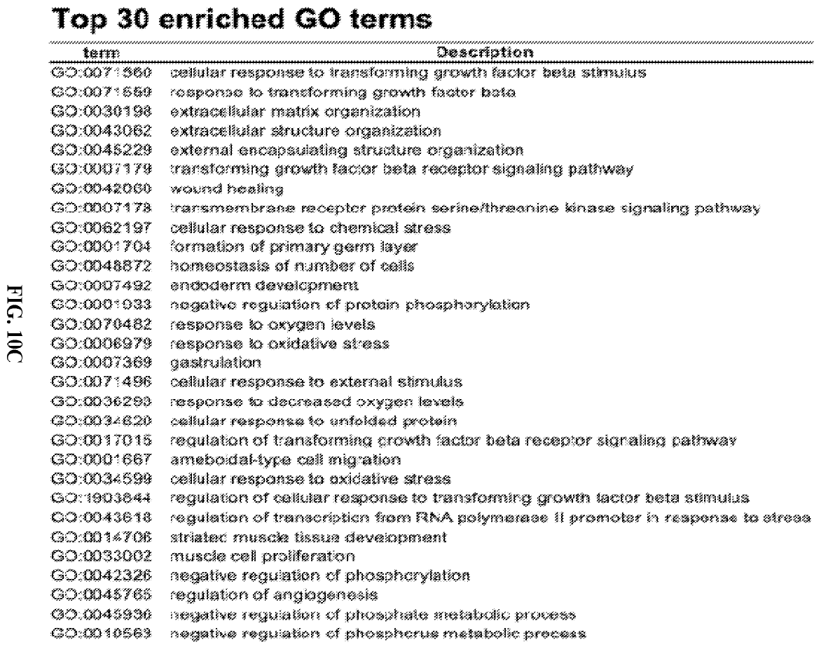

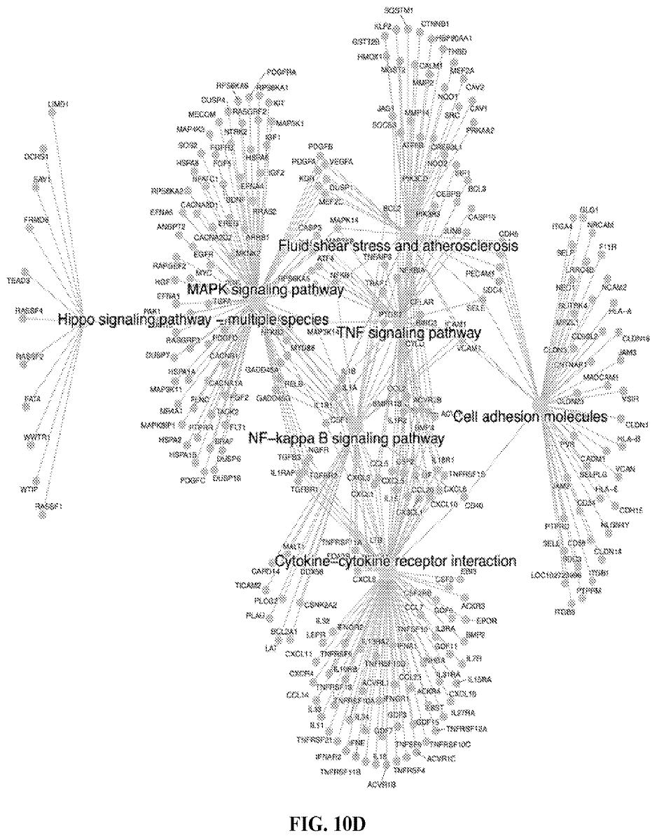

Having established MoNP as an effective targeted delivery vehicle, the potential in formulating a nanodrug aimed at blocking YAP/TAZ dysregulation in activated endothelium was investigated and its efficacy in reducing arterial inflammation and plaque development was assessed. Verteporfin (trade name Visudyne), is a benzoporphyrin derivative and used as a used as a photosensitizer for photodynamic therapy to bind and eliminate abnormal blood vessels. MoNP loaded with Verteporfin (VP) (i.e., MoNP-VP) was prepared. YAP/TAZ-TEAD interactions were assessed, and the formulation was tested in EC (FIG. 9A). MoNP-VP has a hydrodynamic size of 289.4 nm, and approximately 75% of the input VP was loaded into MoNP, with a LE of 16.8% (FIG. 9B). EC treated with MoNP-VP exhibited significantly decreased YAP/TAZ expression and inflammatory responses induced by TNFα, compared to the vehicle control (i.e., MoNP), as evidenced by reductions of CTGF, VCAM1, and ICAM1 expressions and Mo attachment to EC (FIG. 9C-9E). Moreover, RNA-seq profiling was performed to assess the transcriptomic responses of EC to MoNP-VP treatment compared to MoNP, which identified a total of 4,707 DEGs, with 2,333 upregulated and 2,374 downregulated (FIG. 10A). Consistently, MoNP-VP reduced the expressions of atheroprone genes, including VCAM1, ICAM1, SELE, CCL2, and CXCR4, as well as YAP/TAZ-targeted genes such as CTGF, GATA3, and TNS3 (FIG. 9F and FIG. 10B). Additionally, MoNP-VP increased the expressions of atheroprotective genes, including KLF2, KLF4, and NRF2. KEGG pathway enrichment and GO analysis using ClusterProfiler was further performed (FIG. 9G, and FIG. 10C-10D). The results identified TNF signaling pathway, NF-κB signaling pathway, cytokine-cytokine receptor interaction, fluid shear stress and atherosclerosis, and Hippo signaling pathway among the top enriched pathways, indicating MoNP-VP treatment exerted significant effects on the molecular pathways involved in endothelial inflammation and atherosclerosis. These results, collectively, suggested that MoNP-VP effectively suppressed YAP/TAZ activation and subsequent atherogenic responses in EC.

### Example 5: MoNP-VP Treatment Attenuates Arterial Inflammation and Atherosclerosis In Vivo

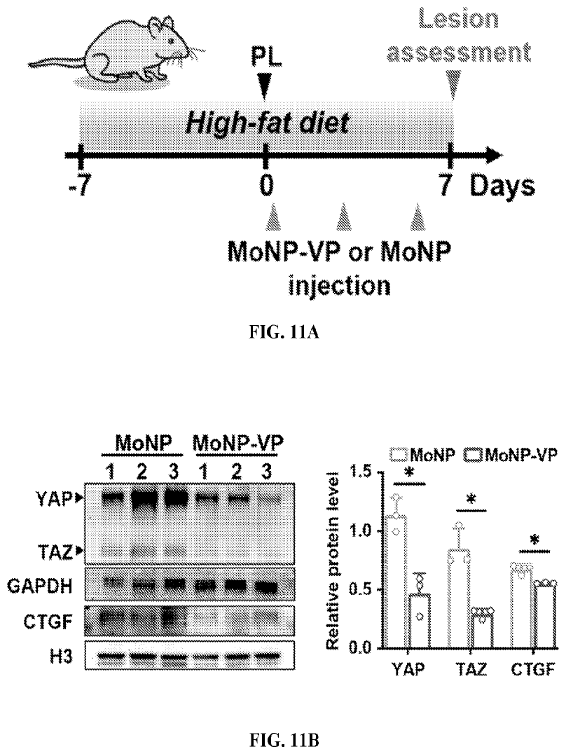

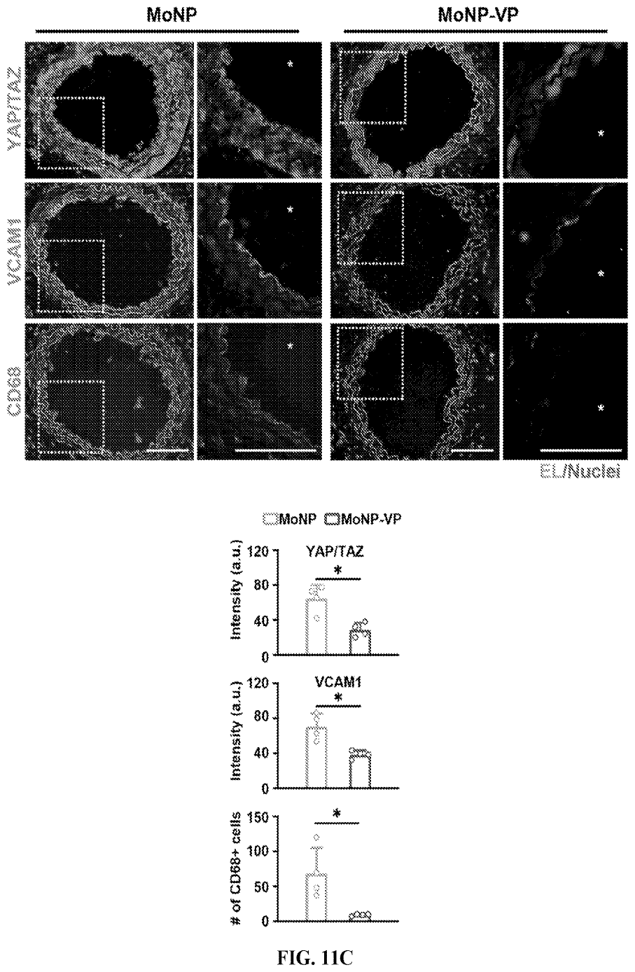

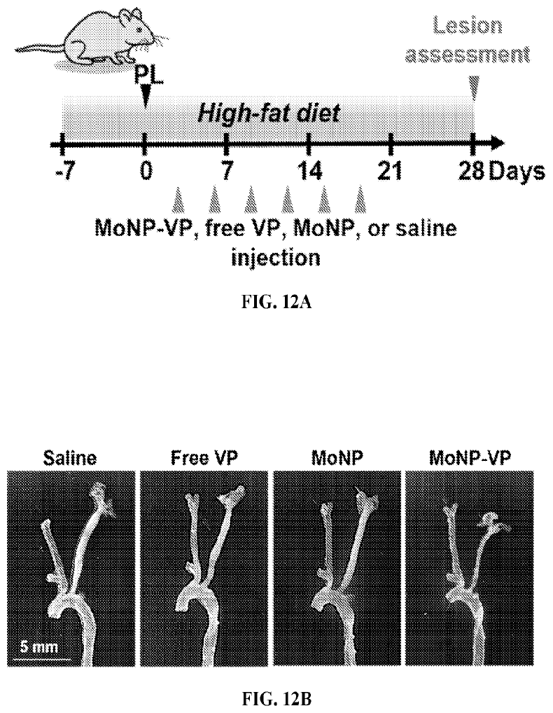

The pharmacological actions of MoNP-VP was further evaluated in mouse arteries. Specifically, the impact of MoNP-VP treatment on YAP/TAZ expression and arterial inflammation was assessed using the PL model. ApoE^{-/-} mice were intravenously administered with MoNP-VP or MoNP at a dose of 2 mg/kg immediately after PL and then every 72 hours for a total of 3 injections (FIG. 11A). The arterial tissue was collected after 1 week after the operation to assess the effects of MoNP-VP on YAP/TAZ expression and macrophage recruitment. Consistent with the in vitro data, the expressions of YAP/TAZ and CTGF in the arterial wall were significantly decreased by MoNP-VP treatment, as compared to MoNP (FIG. 11B). Furthermore, immunofluorescence staining of LCA cross-sections demonstrated that MoNP-VP treatment markedly reduced YAP/TAZ expression throughout the vessel wall, VCAM1 expression in the intima layer, and the number of infiltrated CD68-positive cells, compared to MoNP (FIG. 11C). These findings indicated that VP delivered by MoNP was highly effective in blocking YAP/TAZ expression in vascular cells, subsequently reducing PL-induced arterial inflammation by suppression of endothelial activation and leukocyte recruitment.

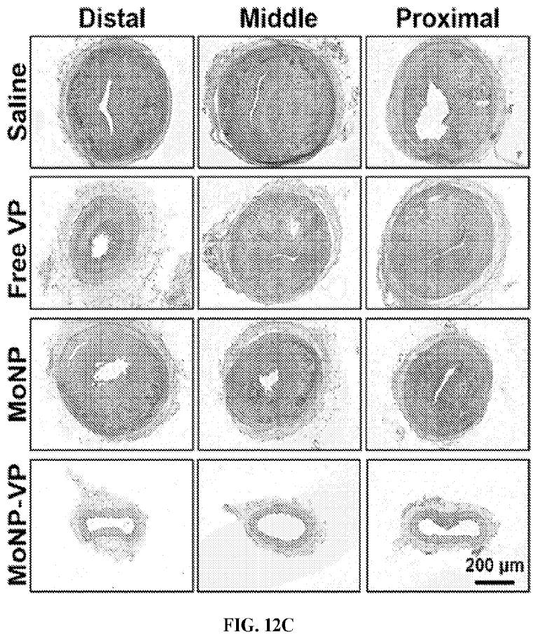

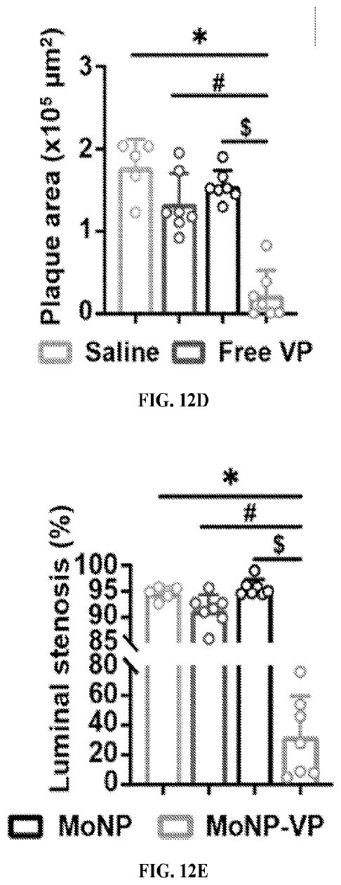

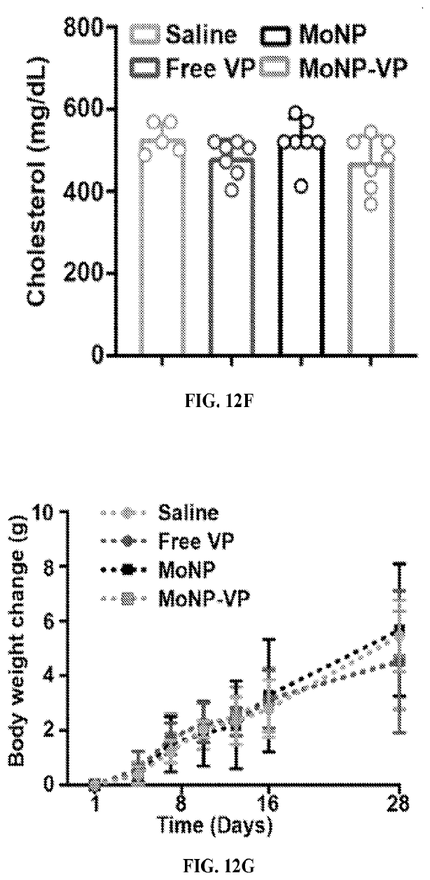

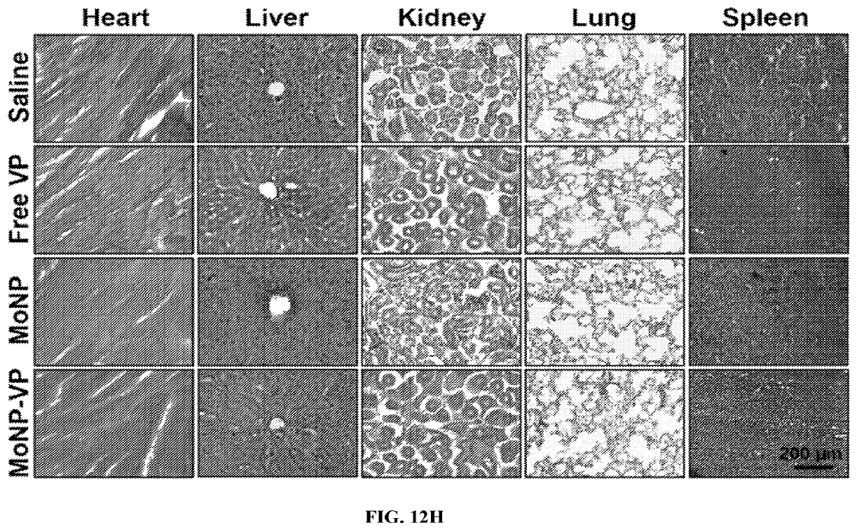

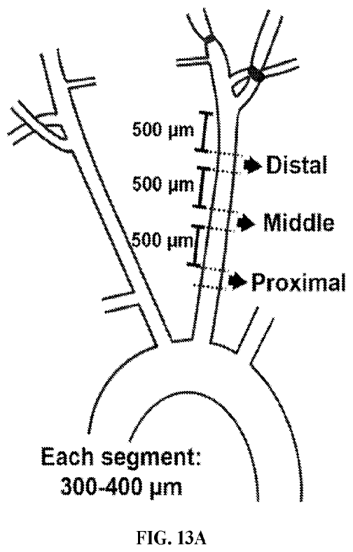

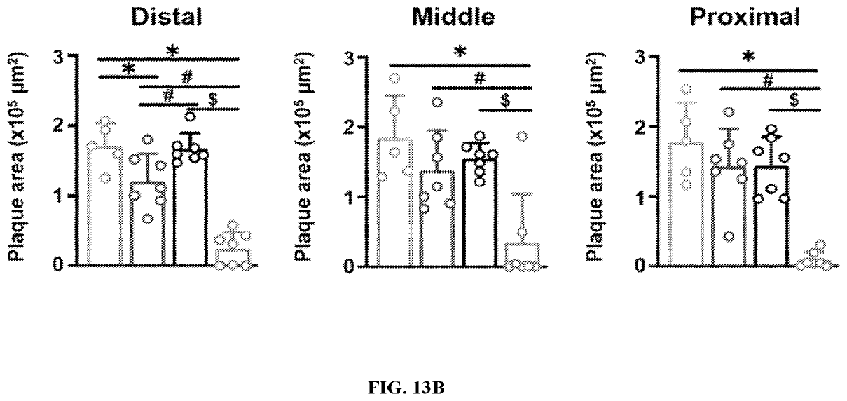

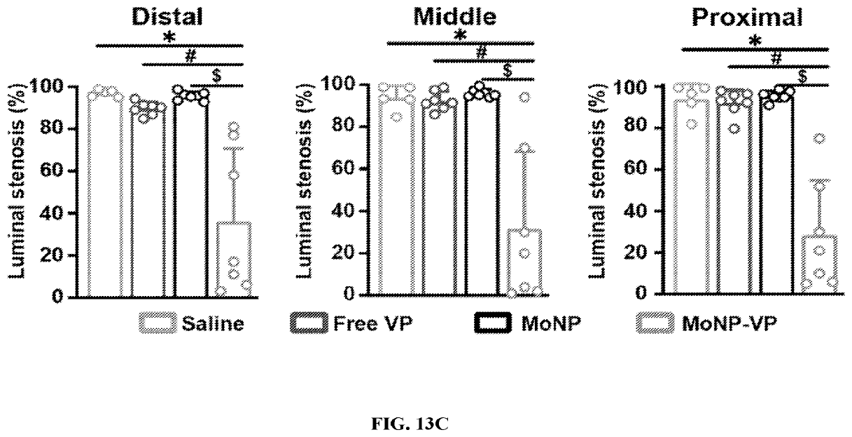

To assess the therapeutic potential of MoNP-VP treatment in plaque development, MoNP-VP, MoNP, free VP, or saline was administered to ApoE^{-/-} mice that underwent PL for a total of 6 injections. Four weeks after the PL procedure, the arterial tissue was harvested and serum to analyze the size of plaque buildup in the LCA and total cholesterol level, respectively (FIG. 12A). Only MoNP-VP treatment, but not free VP or MoNP, was able to reduce the overall size of atherosclerotic lesions in the LCA (FIG. 12B). To quantify plaque size, three segments of the LCA from the proximal end to the carotid bifurcation were analyzed using Oil Red O and hematoxylin staining of LCA cross-sections. Compared to the free VP treatment and controls (i.e., MoNP, saline), MoNP-VP treatment significantly decreased PL-induced carotid atherosclerosis, as evidenced by the reductions of Oil Red O-positive plaque area and percentage of luminal occlusion in the LCA (FIGS. 12C-12E, and FIGS. 13A-13C). These results suggested that MoNP-VP, without photoactivation, effectively suppressed plaque development at a dose of 2 mg/kg and that the targeted delivery strategy significantly improved outcomes compared to the systemic administration of VP, at least within the tested dosing regimen. Additionally, there was no difference in the level of serum cholesterol between MoNP-VP and other groups, suggesting MoNP-VP exerts direct anti-atherosclerotic effects on the affected artery, rather than modulation of blood cholesterol (FIG. 12F).

As the assessment of potential toxicity, the body weight of the mice was monitored throughout the experiment and significant change due to MoNP-VP treatment was not observed (FIG. 12G). Major organs were harvested at the end of the experiment and obvious histopathological alterations were not detected in any of the organs at the tested dose and frequency (FIG. 12H). Additionally, the serum samples were analyzed using a metabolic panel test, which showed that MoNP-VP treatment did not alter levels of blood chemicals and metabolites, including markers indicating liver or kidney damage (Table 2). These findings demonstrated that the MoNP-VP dosage used in this experiment did not elicit apparent side effects in major organs.

TABLE 2
The metabolic panel of mice receiving MoNP-VP

| Tests | MoNP-VP (n = 6) | Reference values |
|---|---|---|
| Albumin (g/dL) | 3.48 ± 0.13 | 2.6-5.4 |
| Alkaline phosphatase (U/L) | 84 ± 8 | 16-200 |
| Alanine aminotransferase | 59.83 ± 30.09 | 22-133 |
| (U/L) | | |
| Total bilirubin (mg/dL) | 0.56 ± 0.15 | 0.1-0.9* |
| Blood urea nitrogen (mg/dL) | 21.33 ± 2.66 | 2-71 |
| Calcium (mg/dL) | 10.20 ± 0.67 | 6.8-11.9* |
| Phosphorus (mg/dL) | 6.2 ± 1.81 | 6.0-11.3* |
| Creatinine (mg/dL) | 0.2 ± 0.0 | 0.1-1.8 |
| Glucose (mg/dL) | 192.33 ± 70.54 | 60-133 |
| Sodium (nmol/L) | 141.33 ± 2.34 | 153-175 |
| Potassium (nmol/L) | 6.38 ± 1.62 | 6.5-9.7* |
| Total Protein (g/dL) | 4.67 ± 0.19 | 4.6-7.3 |

*The reference values for CD1 were utilized in cases where the corresponding values for C57BL6 are unavailable.

### SUMMARY OF EXAMPLES

A promising strategy for treating atherosclerosis is locally blocking inflammatory pathways and cellular events in the arterial wall, potentially minimizing systemic exposure and unwanted side effects compared to systemic routes. Despite its promise, the development of localized atherosclerosis therapies has been hindered by the lack of a plaque-specific delivery vehicle and effective molecular targets. To address this unmet need, a lesion-targeted biomimetic nanodrug, MoNP, packaged with a therapeutic agent, VP, was developed to suppress YAP/TAZ activities specifically within atheroprone arterial regions. The in vitro and in vivo findings have validated that MoNP enhanced the uptake of the payload by inflamed EC while avoiding phagocytic cells, thereby extending their circulation time and achieving lesion-specific delivery. Further, MoNP-mediated delivery of VP effectively reduced YAP/TAZ-mediated inflammatory events in EC and attenuated macrophage infiltration and plaque development in the arteries of ApoE^{-/-} mice, without causing noticeable adverse reactions in major organs. These findings suggested that local inhibition of YAP/TAZ activities using MoNP-VP was a highly effective and safe strategy for treating arterial inflammation and atherosclerosis.

Unlike the traditional NP functionalization that require complex conjugation processes, the disclosed biomimetic platform offers a one-step strategy to coat NP with a lipid bilayer membrane derived from primary Mo, which improved immune evasion and enhanced targeting capabilities. The resulting MoNP displayed multiple targeting moieties of classical Mo including integrins (e.g., very late antigen-4), CCR2, and selectins on their surface, which simultaneously interact with the adhesion molecules (e.g., VCAM1, ICAM1), ligands, and receptor on activated EC to achieve strong adherence. This was supported by the in vitro study showing that Mo membrane cloaking significantly enhanced the internalization of NP into TNFα-stimulated EC compared to untreated EC. The enhanced uptake of MoNP by inflamed EC could be blocked by pretreatment with a VCAM1 antibody, suggesting similar to the homing of circulating Mo, the inflammatory tropism of EC plays a primary role in recruiting MoNP to the diseased artery, alleviating concerns about unintended interactions with healthy vasculature. Furthermore, the selective targeting capability of MoNP to atheroprone vasculature was further validated using the PL model, in which the fluorescent payload of MoNP was found to exclusively accumulate in the early plaque regions of LCA, highlighting its strong potential as an inflammation-targeted drug delivery vehicle.

One advantage of the disclosed MoNP over other EC-targeted delivery vehicles is their potential to reduce the opsonization of nanocarriers by the MPS and extend the retention time of payloads in circulation. The in vitro and in vivo results provided clear evidence for the enhanced immune evasion of MoNP, demonstrating that a significantly greater internalization of uncoated NP by phagocytes compared to MoNP after incubation, and that the plasma of mice receiving MoNP had a considerably higher concentration of fluorescent payload 4 hour-post administration. Modifications of MoNP include increasing the membrane-to-NP ratio, improving membrane coating efficiency, and incorporating “don't eat me” signals (e.g., CD47) or the membrane from myeloid cells with a longer half-life are further contemplated. Additionally, using Mo membrane sourced from the patient's own blood or bone marrow is contemplated and such MoNP would minimize immunogenicity and extend blood resident time, ultimately enhancing therapeutic efficacy.

A critical challenge in treating plaque development and progression lies in the lack of druggable targets linked to inflammatory-fibrotic activities in the arterial wall. Building on the pro-atherogenic roles of YAP/TAZ dysregulation, a therapeutic strategy employing MoNP for targeted delivery of a YAP/TAZ antagonist, was developed herein, which specifically confines YAP/TAZ inhibition to the artery exposed to atherogenic stimuli. Based on its ability to disrupt YAP/TAZ-TEAD interactions and status as an FDA-approved agent, a non-photoactivated VP as a therapeutic payload to treat YAP/TAZ-induced pathologies in atherosclerotic arteries is provided herein. The RNA-seq assay further supported that MoNP treatment effectively suppressed inflammatory responses in EC through dual effects: downregulating YAP/TAZ-associated and atheroprone genes, and upregulating atheroprotective genes. Additionally, the KEGG pathway enrichment analysis of the RNA-seq data revealed that VP delivered by MoNP exerted inhibitory effects primarily on inflammatory pathways associated with atherosclerosis, including Hippo-YAP/TAZ signaling, without affecting those involved in cell necrosis or apoptosis. Collectively, these findings established MoNP-VP as a lesion-targeted, pathway-specific nanodrug that effectively blocks YAP/TAZ functions and mitigated the inflammatory responses in EC while preserving essential functions. These results indicated VP as a therapeutic option in the treatment of atherosclerosis and other inflammatory diseases.

The present disclosure provided evidence highlighting the therapeutic potential of the disclosed targeted VP nanodrug in treating arterial inflammation and atherosclerosis through a light-independent mechanism. Specifically, the animal studies showed that without photoactivation, MoNP-VP treatment at a dose of 2 mg/kg was effective to reduce the expression of YAP/TAZ and VCAM1 in PL-operated arteries and macrophage infiltration. In a long-term experiment, repeated doses of MoNP-VP greatly diminished PL-induced plaque formation without altering the serum lipid profile, whereas the free drug counterpart had little-to-no effect on plaque sizes. This suggested that targeted delivery was more efficient than systemic routes, as a much higher dose of VP may be required for the latter to achieve a therapeutic effect. YAP/TAZ activation and overexpression have also been associated with the dedifferentiation of vascular smooth muscle cells (SMC), as well as the polarization of macrophages towards a proinflammatory M1 phenotype, known to exacerbate plaque progression. In this regard, the locally released VP in the diseased arterial wall could potentially exert additional beneficial effects on suppressing the pathological phenotypes of SMC and reducing the number of M1 macrophages, and could further improve the treatment outcome for atherosclerosis.

Further, histology staining and serum biochemistry analyses conducted herein, revealed no significant pathological characteristics in major organs attributable to MoNP-VP treatment. Moreover, the body weight changes observed in all experimental mice remained within the normal range. These findings suggested that the dose utilized herein was well-tolerated in mice, and that the MoNP-VP treatment of atherosclerosis was both safe and therapeutically effective in alleviating arterial inflammation and plaque development.

In summary, disclosed herein is an inflammation-targeted drug delivery system utilizing nanocarriers coated with Mo membrane. This MoNP platform greatly enhanced the immune evasion of the nanocarriers and boosted their targeting to atheroprone vasculature while avoiding healthy blood vessels. MoNP employed to deliver VP, achieved lesion-targeted and pathway-specific suppression of YAP/TAZ, leading to robust anti-inflammatory and anti-atherosclerotic effects without requiring photoactivation. Furthermore, MoNP-mediated delivery maximized the therapeutic efficacy of VP while minimizing its organ toxicity. The disclosed delivery system provides biomimicry and nanomedicine for more effective and tailored treatment options for atherosclerosis and other inflammatory diseases, propelling the advancement of personalized and precision medicine.

What is claimed is:

1. A lipid nanoparticle complex comprising (a) a nanoparticle and (b) a lipid membrane encapsulating the nanoparticle.

2. The lipid nanoparticle complex of claim 1, wherein the lipid membrane comprises a monocyte plasma membrane.

3. The lipid nanoparticle complex of claim 1, wherein the lipid membrane does not comprise a cytosolic protein.

4. The lipid nanoparticle complex of claim 1, wherein the lipid membrane comprises at least one membrane protein.

5. The lipid nanoparticle complex of claim 4, wherein the membrane protein is selected from an adhesion protein, a glycoprotein, and a type I transmembrane protein.

6. The lipid nanoparticle complex of claim 1, further comprising at least one active pharmaceutical agent.

7. The lipid nanoparticle complex of claim 6, wherein the active pharmaceutical agent comprises a YAP/TAZ inhibitor.

8. The lipid nanoparticle complex of claim 7, wherein the YAP/TAZ inhibitor is selected from thiazovivin, cucurbitacin I, dasatinib, fluvastatin, pazopanib, verteporfin (VP) or any combination thereof.

9. The lipid nanoparticle complex of claim 1, wherein the complex has an average hydrodynamic diameter of from about 200 to about 700 nm, about 300 to about 600 nm, or from about 200 to about 300 nm.

10. The lipid nanoparticle complex of claim 9, wherein the complex has an average hydrodynamic diameter of about 280 nm.

11. The lipid nanoparticle complex of claim 1, wherein the complex has a surface charge of about −15 to −20 mV.

12. The lipid nanoparticle complex of claim 11, wherein the complex has a surface charge of about −17.5 mV.

13. A pharmaceutical composition comprising a lipid nanoparticle complex of claim 8, and a carrier or excipient.

14. A method of producing a lipid nanoparticle complex comprising:
(a) forming a nanoparticle comprising an active pharmaceutical agent;
(b) isolating a plasma membrane from a monocyte;
(c) contacting the plasma membrane of (b) with the nanoparticle of (a) to form the lipid nanoparticle complex.

15. The method of claim 14, wherein the monocyte is isolated from a bone marrow of a subject.

16. The method of claim 14, wherein forming the nanoparticle comprises preparing a PLGA emulsion.

17. A method of treating atherosclerosis in a subject in need thereof, the method comprising administering to the subject an effective amount of a lipid nanoparticle of claim 8, or of the pharmaceutical composition of claim 13.

18. The method of claim 17, wherein the pharmaceutical composition is administered intravenously to atherosclerotic lesions.

19. The method of claim 17, wherein the subject is a human.

20. A lipid nanoparticle of claim 8, or the pharmaceutical composition of claim 13 for use in the treatment of atherosclerosis.


SEQUENCE LISTING (8 sequences)

SEQ ID NO: 1 - DNA, 20 bp, synthetic construct
  source 1..20: mol_type = other DNA
ggcagagtacgcaaacactt

SEQ ID NO: 2 - DNA, 20 bp, synthetic construct
  source 1..20: mol_type = other DNA
acaggattttcggagcagga

SEQ ID NO: 3 - DNA, 20 bp, synthetic construct
  source 1..20: mol_type = other DNA
cttgagggcacctacctctg

SEQ ID NO: 4 - DNA, 20 bp, synthetic construct
  source 1..20: mol_type = other DNA
cattatgactgcggctgcta

SEQ ID NO: 5 - DNA, 19 bp, synthetic construct
  source 1..19: mol_type = other DNA
gcaccaccaactgcttagc

SEQ ID NO: 6 - DNA, 20 bp, synthetic construct
  source 1..20: mol_type = other DNA
atgatgttctggagagcccc

SEQ ID NO: 7 - DNA, 20 bp, synthetic construct
  source 1..20: mol_type = other DNA
ggcccagacccaactatgat

SEQ ID NO: 8 - DNA, 20 bp, synthetic construct
  source 1..20: mol_type = other DNA
aggaggcgttgtcattggta